SEQUENCE LISTING (2 sequences)

SEQ ID NO: 1 - DNA, 20 bp, Artificial Sequence
  Description of Artificial Sequence oligonucleotide primer
gaccgatccagcctccgcgg

SEQ ID NO: 2 - DNA, 20 bp, Artificial Sequence
  Description of Artificial Sequence oligonucleotide primer
cgtgtgtcccgcggggagaa

(19) United States
(12) Patent Application Publication
(10) Pub. No.: US 2005/0037335 A1
(43) Pub. Date: Feb. 17, 2005

(54) Method for identifying novel transcriptional regulatory proteins

(76) Inventor: Wolfgang Hillen, Uttenreuth (DE)

Correspondence Address: LAHIVE & COCKFIELD, LLP., 28 STATE STREET, BOSTON, MA, 02109, US

(21) Appl. No.: 10/108,524

(22) Filed: Mar. 27, 2002

Related U.S. Application Data
(62) Division of application No. 09/587,623, filed on Jun. 5, 2000, now abandoned.
(60) Provisional application No. 60/137,671, filed on Jun. 4, 1999.

(30) Foreign Application Priority Data
Nov. 7, 2000 (DE) DE 19851413 A 1

(51) Int. Cl. C12Q 1/70; C12Q 1/68; C12N 15/86

(52) U.S. Cl. 435/5; 435/6; 435/456

(57) ABSTRACT

A method for identifying transcriptional regulatory proteins that modulate the transcription of a gene of interest is disclosed. In one embodiment, the method comprises introducing into a cell a nucleic acid molecule comprising three central components: 1) a polynucleotide (e.g., DNA) encoding a transcriptional regulatory protein; 2) an indicator gene which is responsive to, e.g., under the transcriptional control of, the gene regulatory sequences of a gene of interest which bind to the transcriptional regulatory protein; and 3) a selectable marker gene. The transcriptional regulatory protein binds to the gene regulatory sequences and either activates or inhibits transcription. A protein is identified as a modulator of the transcription of the gene of interest by detecting a signal generated by the indicator gene.

### RELATED APPLICATIONS

This application claims priority to U.S. Provisional Application No. 60/137,671, filed on Jun. 4, 1999, incorporated herein in its entirety by this reference.

### BACKGROUND OF THE INVENTION

The Tn10-encoded Tet repressor (TetR) protein regulates the expression of tetracycline resistance genes in gram negative bacteria, e.g. Escherichia coli, in a tetracycline (Tc) dependent fashion (reviewed in Hillen & Berens, 1994). In the absence of Tc, a TetR protein dimer binds to operator sequences (tetO) and inhibits expression of the tetracyline resistance gene (tetA). When the inducer Tc enters the cell and binds to TetR, the affinity for tetO is reduced and TetR dissociates from tetO, allowing expression of tetA. The crystal structures of the TetR-[Mg-Tc]⁺ complex (Hinrichs et al., 1994; Kisker et al., 1995) and free TetR (Orth et al., 1998), and analysis of non-inducible TetR mutants (Müller et al., 1995), imply that the binding of Tc induces conformational changes in TetR. Dimerization of TetR is mediated by a four helix bundle, and residues which determine the dimerization specificity have been identified (Schnappinger et al., 1998). This has led to TetR based regulators which cannot heterodimerize.

TetR-based transcription activators have been developed which allow inducible expression of appropriately modified genes in a tetracycline dependent mode (Gossen & Bujard, 1992; Gossen et al., 1995) in various cellular systems of mammalian (Gossen & Bujard, 1992), plant (Weinmann et al., 1994; Zeidler et al., 1996) and amphibian (Camacho-Vanegas et al., 1998) origin, as well as in whole organisms including fungi (Gari et al., 1997), plants (Weinmann et al., 1994), Drosophila (Bello et al., 1998), mice (Kistner et al., 1996; Efrat et al., 1995; Ewald et al., 1996) and rats (Fishman et al., 1994; Harding et al., 1998).

Tetracycline controlled transactivators (tTA) are fusions between TetR and proper domains of transcriptional activators. In one such fusion protein, a major portion of the Herpes simplex virus protein 16 (VP16) was fused at the level of DNA to TetR. Yet, other tTA's demonstrate a graded transactivation potential resulting from connecting different combinations of minimal activation domains to the C-terminus of TetR (Baron et al., 1997). These chimeric “tetracycline controlled transactivators” (tTA) allow one to regulate the expression of genes placed downstream of minimal promoter-tetO fusions (Pₜₑₜ). In absence of Tc Pₜₑₜ is activated whereas in presence of the antibiotic activation of Pₜₑₜ is prevented.

A “reverse tetracycline controlled transactivator” (rtTA) was developed which binds operator DNA only in the presence of some tetracycline derivatives such as doxycycline (Dox) or anhydrotetracycline (ATc), and thus activates Pₜₑₜ upon addition of Dox (Gossen et al., 1995). Both tTA and rtTA are widely used to regulate gene expression in various systems (for review see Freundlieb et al., 1997).

Despite widespread use of Tet systems in academic and industrial research, as well as in some technical processes such as high throughput screening and fermentation, there are limitations which prevent their use in a number of areas because of the specific properties of the transactivators, and of the inducing effector substances.

These limitations concern particularly:

the residual affinity of rtTA to tetO sequences in the absence of the inducer;
the relatively low susceptibility of rtTA towards Dox;
the interdependence between different domains of tTA and rtTA, that can affect the specificity of transactivator/operator interaction;
the stability of tTA and rtTA in different eukaryotic systems;
the relatively narrow temperature optimum of tTA/rtTA function;
the antibiotic activity of some of the best effector molecules; and
the restriction of effectors to substances of the tetracycline family.

For example, the known rtTA described above has retained a residual affinity to tetO in the absence of doxycycline (Dox). This can lead to an intrinsic basal activity of rtTA responsive promoters, and indeed such increased basal levels of transcription have been observed in mammalian cell lines as well as in S. cerevisiae. Tc controlled expression using tTA and rtTA in S. cerevisiae has been published (Gallego et al., 1997; Gan et al., 1997; Belli et al., 1998a; Belli et al., 1998b; Nagahashi et al., 1998; Nakayama et al., 1998; Colomina et al., 1999). Gene regulation was achieved with tTA showing high expression of lacZ and low basal activities (Bari et al., 1997). In contrast, rtTA did not regulate expression in response to Tc due to extremely high basal expression, leaving no room for apparent induction of gene expression. Thus, an additional regulated repressor was introduced to lower the basal expression (Belli et al., 1998). Only this dual control system previously yielded reasonable induction factors in S. cerevisiae. In addition, the known rtTA is fully induced only at relatively high levels of Dox.

Moreover, it appears that active rtTA proteins cannot be synthesized in a number of systems including B-cells in transgenic (tg) mice, Drosophila melanogaster, and plants. Whether this is due to instabilities at the level of RNA or protein, or both is not entirely clear.

The known transactivators also exhibit a rather narrow temperature optimum. In mammalian systems, this does not pose a particular problem. By contrast, applying Tet regulation to plants will require an expanded temperature tolerance of transactivators.

Previously, the most efficient way of producing TetR variants was based on random or directed mutagenesis, followed by screening procedures that relied on TetR function in E. coli (Helbl & Hillen, 1998; Helbl et al., 1998; Müller et al., 1995; Hecht et al., 1993; Wissmann et al., 1991). TetR variants identified in this way were subsequently converted to tTA and/or rtTA fusion proteins whose properties were examined in eukaryotic systems. Frequently, the properties of TetR variants as identified in E. coli would not correlate with those of the corresponding tTA or rtTAs in eukaryotic cells. The main reasons for these inconsistencies are: (a) fusion of activator domains to TetR variants or introduction of mutations, e.g., mutations that confer the reverse phenotype, may negatively affect the overall function of the respective TetR variant; (b) the properties of tTA/rtTA's such as stability or the interaction with operator sequences is affected by differences in the cellular environment between E. coli and various eukaryotic systems; and (c) tetracycline and many of its derivatives are toxic in prokaryotes where they act primarily to inhibit protein biosynthesis, and thus limit screening procedures to sublethal concentrations of the effector molecule. By contrast, tetracyclines are tolerated at higher concentrations in eukaryotic cells.

It is therefore necessary to examine fully the useful sequence space of the Tet repressor. To this end, it is desirable to develop a screening method which is capable of rapidly and efficiently identifying novel variants of tTA and rtTA out of large pools of candidates produced by random, semi-random and directed mutagenesis.

Optimal application of tTA's and rtTA's in different eukaryotic systems will require the development of transactivators that are specifically adapted to defined tasks. Therefore, screening systems that are capable of identifying tTA/rtTA phenotypes directly in eukaryotes like yeast or other fungi will constitute a significant improvement over the current screening technology for the following reasons:
the phenotypes identified will directly reflect the properties of the transactivating fusion protein (TetR fused to an activation domain) in an eukaryotic system;
mutagenesis can be performed throughout the gene encoding the entire transactivator;
mutations within the activation domain can be included in the analysis; and
using yeast or other fungal systems will result in screening efficiencies that are comparable to those obtained in E. coli.

### SUMMARY OF THE INVENTION

In one aspect, the invention is a method for identifying a transcriptional regulatory protein that modulates transcription of a gene of interest. The method comprises:

providing a cell comprising a nucleic acid molecule comprising first, second and third polynucleotides, wherein:
the first polynucleotide encodes a fusion protein comprising a first polypeptide in operative linkage to a second polypeptide, wherein the first polypeptide is derived from the DNA binding domain of a first protein that binds to a regulatory sequence of the gene of interest, and the second polypeptide is derived from the transcriptional regulatory domain of a second protein;
the second polynucleotide comprises an indicator gene, the expression of which is responsive to the fusion protein; and
the third polynucleotide comprises a selectable marker gene; and

detecting a signal generated by the indicator gene to thereby identify the fusion protein as a transcriptional regulatory protein that modulates transcription of the gene of interest. In a preferred embodiment, the cell is further treated with a modulator molecule, or analog thereof, wherein binding of the first polypeptide to the regulatory sequence of the gene of interest is controlled by the modulator molecule or analog thereof.

Another aspect of the invention is a method for identifying a Tet repressor-based regulatory protein that modulates transcription of a gene of interest. The method comprises:

providing a cell comprising a nucleic acid molecule comprising first, second and third polynucleotides, wherein:
the first polynucleotide encodes a Tet repressor-based regulatory protein comprising a first polypeptide in operative linkage to a second polypeptide, wherein the first polypeptide is derived from a Tet repressor protein that binds to a regulatory sequence of the gene of interest, and wherein the binding to the regulatory sequence is controlled by a modulator molecule, or an analog thereof, and the second polypeptide is derived from the transcriptional regulatory domain of a second protein;
the second polynucleotide comprises an indicator gene, the expression of which is responsive to the Tet repressor-based regulatory protein; and
the third polynucleotide comprises a selectable marker gene;

treating the cell with the modulator molecule, or an analog thereof; and

detecting a signal generated by the indicator gene to thereby identify the Tet repressor-based regulatory protein as a modulator of transcription of the gene of interest.

Yet, another aspect of the invention is a method for identifying a Tet repressor-based regulatory protein that modulates transcription. The method comprises:

providing a cell comprising a nucleic acid molecule comprising first, second and third polynucleotides, wherein:
the first polynucleotide encodes a Tet repressor-based regulatory protein comprising a first polypeptide in operative linkage to a second polypeptide, wherein the first polypeptide is derived from a Tet repressor protein that binds to regulatory sequences derived from the Tet operator, and wherein the binding to the Tet operator sequences is controlled by tetracycline, or an analog thereof, and the second polypeptide is derived from the transcription activation domain of a second protein;
the second polynucleotide comprises an indicator gene, the expression of which is regulated by sequences derived from the Tet operator; and
the third polynucleotide comprises a selectable marker gene;

treating the cell with tetracycline, or an analog thereof; and

detecting a signal generated by the indicator gene to thereby identify the Tet repressor-based regulatory protein as a modulator of transcription.

A further aspect of the invention is a method for identifying a compound that is capable of modulating a transcriptional regulatory protein that modulates transcription of a gene of interest. The method comprises:

providing a cell comprising a nucleic acid molecule comprising first, second, and third polynucleotides, wherein:
the first polynucleotide encodes a fusion protein comprising a first polypeptide in operative linkage to a second polypeptide, wherein the first polypeptide is derived from the DNA binding domain of a first protein that binds to a regulatory sequence of a gene of interest, and wherein the binding to the regulatory sequence is controlled by a modulator compound, and said second polypeptide is derived from a transcription activation domain of a second protein;
the second polynucleotide comprises an indicator gene, the expression of which is responsive to the fusion protein; and
the third polynucleotide comprises a selectable marker gene;

treating the cell with the compound; and

detecting a signal generated by the indicator gene to thereby identify the compound as a modulator of the transcriptional regulatory protein.

Another aspect of the invention is a recombinant vector which comprises:

a nucleic acid molecule comprising first, second and third polynucleotides, wherein:
the first polynucleotide encodes a fusion protein comprising a first polypeptide in operative linkage to a second polypeptide, wherein the first polypeptide is derived from the DNA binding domain of a first protein that binds to a regulatory sequence of a gene of interest, and the second polypeptide is derived from the transcriptional regulatory domain of a second protein;
the second polynucleotide comprises an indicator gene, the expression of which is responsive to the fusion protein; and
the third polynucleotide comprises a selectable marker gene.

Yet another aspect of the invention is a recombinant vector which comprises:

a nucleic acid molecule comprising first, second and third polynucleotides, wherein:
the first polynucleotide encodes a Tet repressor-based regulatory protein comprising a first polypeptide in operative linkage to a second polypeptide, wherein the first polypeptide is derived from a Tet repressor protein that binds to a regulatory sequence of said gene of interest, and wherein the binding to the regulatory sequence is controlled by a modulator molecule, or an analog thereof, and the second polypeptide is derived from the transcriptional regulatory domain of a second protein;
the second polynucleotide comprises an indicator gene, the expression of which is responsive to the Tet repressor-based regulatory protein; and
the third polynucleotide comprises a selectable marker gene.

A further aspect of the invention is a recombinant vector which comprises:

a nucleic acid molecule comprising first, second and third polynucleotides, wherein:
the first polynucleotide encodes a Tet repressor-based regulatory protein comprising a first polypeptide in operative linkage to a second polypeptide, wherein the first polypeptide is derived from a Tet repressor protein that binds to regulatory sequences derived from the Tet operator, and wherein the binding is controlled by tetracycline, or an analog thereof, and the second polypeptide is derived from the transcription activation domain of a second protein;
the second polynucleotide comprises an indicator gene, the expression of which is regulated by sequences derived from the Tet operator; and
the third polynucleotide comprises a selectable marker gene.

Accordingly, a further aspect of the invention pertains to host cells transformed with a recombinant vector of the invention. The host cell can be a prokaryotic cell, a eukaryotic cell, preferably a yeast cell or a mammalian cell.

One aspect of the invention is a method for identifying a polynucleotide gene regulatory sequence that binds to a Tet repressor-based regulatory protein of interest. The method comprises:

providing a cell comprising a nucleic acid molecule comprising first, second and third polynucleotides, wherein:
the first polynucleotide encodes a Tet repressor-based regulatory protein comprising a first polypeptide in operative linkage to a second polypeptide, wherein the first polypeptide is derived from a Tet repressor protein that binds to gene regulatory sequences derived from the Tet operator, and wherein said binding is controlled by tetracycline, or an analog thereof, and the second polypeptide is derived from the transcription activation domain of a second protein;
the second polynucleotide comprises an indicator gene, the expression of which is modulated by gene regulatory sequences derived from the Tet operator; and
the third polynucleotide comprises a selectable marker gene;

treating the cell with tetracycline, or an analog thereof; and

detecting a signal generated by the indicator gene to thereby identify the gene regulatory sequences as binding the Tet repressor-based regulatory protein and modulating gene transcription.

Another aspect of the invention is a method for identifying a polynucleotide gene regulatory sequence that binds to a transcriptional regulatory protein of interest. The method comprises:

providing a cell comprising a nucleic acid molecule comprising first, second and third polynucleotides, wherein:
the first polynucleotide encodes a fusion protein comprising a first polypeptide in operative linkage to a second polypeptide, wherein the first polypeptide is derived from the DNA binding domain of a first protein that binds to gene regulatory sequences of a gene of interest, and the second polypeptide is derived from the transcriptional regulatory domain of a second protein;
the second polynucleotide comprises an indicator gene, the expression of which is modulated by the gene regulatory sequences of the gene of interest; and
the third polynucleotide comprises a selectable marker gene; and

detecting a signal generated by the indicator gene to thereby identify the gene regulatory sequences as binding the fusion protein and modulating gene transcription.

### BRIEF DESCRIPTION OF THE DRAWINGS

FIG. 1 is a graph depicting the rtTA dependent GFP fluorescence in S. cerevisiae in dependence of Tc and doxycycline (Dox).

FIG. 2 is a graph depicting the rtTA dependent GFP fluorescence in S. cerevisiae in dependence of doxycycline (Dox).

FIG. 3 is a graph depicting the rtTA-dependent luciferase expression in HeLa cells in dependence of Tc and/or Dox.

FIG. 4 is a graph depicting the tTA dependent luciferase expression in transiently transfected human epithelial cells in dependence of Tc and/or Dox.

FIG. 5 is a graph depicting the contribution of various mutations in rtTA-34R to the reverse phenotype.

FIG. 6 is a graph depicting the doxycycline-dependent regulation of luciferase by rtTA and rtTA-34R-FFFF in stably transfected HeLa cells.

FIG. 7 is a gel depicting the mobility change of tet operator DNA in presence of rtTA2 and rtTA2-34R.

FIG. 8 depicts the nucleic acid sequence encoding the parent rtTA protein.

FIG. 9 depicts the nucleic acid sequence encoding the parent tTA protein.

### DETAILED DESCRIPTION OF THE INVENTION

The present invention provides methods for the screening and identification of transcriptional regulatory proteins that modulate the transcription of a gene of interest. In addition, the present invention provides a method for the identification of compounds that modulate the activity of a transcriptional regulatory protein of interest. The present invention also provides for a method for the identification of gene regulatory sequences that specifically bind to a transcriptional regulatory protein of interest, thereby mediating the action of the transcriptional regulatory protein on the transcription of an adjacent gene. Compositions of matter, such as novel recombinant vector constructs and novel recombinant host cells are also encompassed in the present invention.

In an advantageous embodiment, the invention provides methods for the screening and identification of Tet repressor-based regulatory proteins that modulate transcription of a gene of interest. In particular, the methods of the invention enable an exhaustive examination of the sequence space of TetR as well as of its fusion products in a eukaryotic screening system that reveals, with high probability, novel alleles that encode significantly improved variants of transactivators (tTA, rtTA) as well as transcriptional silencers (tTS). In certain embodiments of the invention, sequence libraries encoding a large number of tTA variants are screened.

Improved transactivators in accordance with the invention include those with increased temperature tolerance. For example, in accordance with one embodiment of the invention, appropriate fungal systems are used in order to identify tTA/rtTA variants having the desired temperature tolerance properties.

In accordance with the invention, mutagenesis of tTA encoding sequences facilitates the identification of transactivators that interact differentially with different effector molecules. For example, mutagenesis can be restricted to portions of the sequence responsible for forming the effector binding pocket. Such properties could be exploited to control different genes via specific sets of transactivators and effectors (see Baron et al., 1999). Modification of the effector binding pocket is most likely a prerequisite for the detection of tetracyclines that are not deposited in bone tissue. For gene therapy, it will be advantageous to use transactivators that are insensitive toward tetracyclines used in human medicine.

Full effector function at Dox concentrations of 10 to 30 ng/ml, as with tTA, is highly desirable, particularly in experiments involving transgenic animals or in gene therapy. Accordingly, the present invention provides for screening for rtTA variants with increased sensitivity towards Dox.

The invention also provides for the identification of new effector molecules for tTA and rtTA. For example, effector substances that fully induce rtTA at lower concentrations can be identified. The screening methods in accordance with the invention facilitate the examination of substance libraries, advantageously in a high throughput format, for new effectors with superior effector properties and negligible antibiotic activity. Candidates for screening include:
tetracyclines that have lost antibiotic activity;
tetracyclines that mediate rtTA activation at low concentrations;
tetracyclines that may not deposit in bone tissue;
tetracyclines with improved tissue penetration properties;
tetracycline antagonists; and
non-tetracycline compounds that can serve as effectors for tTA and/or rtTA.
   Definitions

Before further description of the invention, certain terms employed in the specification, examples and appended claims are, for convenience, collected here.

The term “compound” as used herein (e.g., as in “modulator compound,” or “test compound”) is meant to include both exogenously added test compounds and peptides endogenously expressed from a peptide library. For example, in certain embodiments, the host cell also produces the test compound which is being screened. For instance, the host cell can produce. e.g., a test polypeptide, a test nucleic acid and/or a test carbohydrate which is screened for its ability to modulate the activity of the transcriptional regulatory protein. In such embodiments, a culture of such reagent cells will collectively provide a library of potential effector molecules and those members of the library which either stimulate or inhibit the activity of the transcriptional regulatory protein can be selected and identified.

The term “derived from” is intended to mean that a sequence is identical to or modified from another sequence. Polypeptide or protein derivatives include polypeptide or protein sequences that differ from the sequences described or known in amino acid sequence, or in ways that do not involve sequence, or both, and still preserve the activity of the polypeptide or protein. Derivatives in amino acid sequence are produced when one or more amino acids is substituted with a different natural amino acid, an amino acid derivative or non-native amino acid. In certain embodiments protein derivatives include naturally occurring polypeptides or proteins, or biologically active fragments thereof, whose sequences differ from the wild type sequence by one or more conservative amino acid substitutions, which typically have minimal influence on the secondary structure and hydrophobic nature of the protein or peptide. Derivatives may also have sequences which differ by one or more non-conservative amino acid substitutions, deletions or insertions which do not abolish the biological activity of the polypeptide or protein.

Conservative substitutions (substituents) typically include the substitution of one amino acid for another with similar characteristics (e.g., charge, size, shape, and other biological properties) such as substitutions within the following groups: valine, glycine; glycine, alanine; valine, isoleucine; aspartic acid, glutamic acid; asparagine, glutamine; serine, threonine; lysine, arginine; and phenylalanine, tyrosine. The non-polar (hydrophobic) amino acids include alanine, leucine, isoleucine, valine, proline, phenylalanine, tryptophan and methionine. The polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid.

The polypeptides and proteins of this invention may also be modified by various changes such as insertions, deletions and substitutions, either conservative or nonconservative where such changes might provide for certain advantages in their use.

In other embodiments, derivatives with amino acid substitutions which are less conservative may also result in desired derivatives, e.g., by causing changes in charge, conformation and other biological properties. Such substitutions would include, for example, substitution of hydrophilic residue for a hydrophobic residue, substitution of a cysteine or proline for another residue, substitution of a residue having a small side chain for a residue having a bulky side chain or substitution of a residue having a net positive charge for a residue having a net negative charge. When the result of a given substitution cannot be predicted with certainty, the derivatives may be readily assayed according to the methods disclosed herein to determine the presence or absence of the desired characteristics.

Derivatives within the scope of the invention also include polynucleotide derivatives. Polynucleotide or nucleic acid derivatives differ from the sequences described or known in nucleotide sequence. For example, a polynucleotide derivative may be characterized by one or more nucleotide substitutions, insertions, or deletions.

The term “DNA binding protein” is intended to include any protein, or functional domain thereof, that specifically interacts with a cognate DNA sequence, or response element, within the regulatory sequences of a gene. The DNA binding domains of transcriptional regulatory proteins can be classified into structural families which include, but are not limited to, basic helix-loop-helix domains, leucine zipper domains, zinc finger domains, and helix-turn-helix domains/homeodomains. A fusion protein of the present invention includes a polypeptide comprising a DNA binding protein, or a functional DNA binding domain thereof. The recognition and binding of a DNA binding protein to its cognate DNA sequence can be regulated by conformational changes in the DNA binding protein itself conferred by the binding of a modulator molecule or ligand. Similarly, the conformation of the cognate DNA sequence within the chromatin, e.g., organized into nucleosome, also influences the binding of a DNA binding protein to its cognate DNA sequence.

The term “gene regulatory sequences” is intended to include the DNA sequences that control the transcription of an adjacent gene. Gene regulatory sequences include, but are not limited to, promoter sequences that are found in the 5′ region of a gene proximal to the transcription start site which bind RNA polymerase to initiate transcription. Gene regulatory sequences also include enhancer sequences which can function in either orientation and in any location with respect to a promoter, to modulate the utilization of a promoter. Transcriptional control elements include, but are not limited to, promoters, enhancers, and repressor and activator binding sites. The gene regulatory sequences of the present invention contain binding sites for transcriptional regulatory proteins. In a preferred embodiment, gene regulatory sequences comprise sequences derived from the tet operator (tetO) which bind tet repressor proteins.

As used herein, a “host cell” includes any cultivatable cell that can be modified by the introduction of heterologous DNA. Preferably, a host cell is one in which a transcriptional regulatory protein can be stably expressed, post-translationally modified, localized to the appropriate subcellular compartment, and made to engage the appropriate transcription machinery. The choice of an appropriate host cell will also be influenced by the choice of detection signal. For example, reporter constructs, as described above, can provide a selectable or screenable trait upon activation or inhibition of gene transcription in response to a transcriptional regulatory protein; in order to achieve optimal selection or screening, the host cell phenotype will be considered.

A host cell of the present invention includes prokaryotic cells and eukaryotic cells. Prokaryotes include gram negative or gram positive organisms, for example, E. Coli or Bacilli. Suitable prokaryotic host cells for transformation include, for example, E. coli, Bacillus subtilis, Salmonella typhimurium, and various other species within the genera Pseudomonas, Streptomyces, and Staphylococcus. Eukaryotic cells include, but are not limited to, yeast cells, plant cells, fungal cells, insect cells (e.g., baculovirus), mammalian cells, and the cells of parasitic organisms, e.g., trypanosomes.

As used herein, the term “yeast” includes not only yeast in a strict taxonomic sense, i.e., unicellular organisms, but also yeast-like multicellular fungi of filamentous fungi. Exemplary species include Kluyverei lactis, Schizosaccharomyces pombe, and Ustilaqo maydis, with Saccharomyces cerevisiae being preferred. Other yeast which can be used in practicing the present invention are Neurospora crassa, Aspergillus niger, Aspergillus nidulans, Pichia pastoris, Candida tropicalis, and Hansenula polymorpha.

Mammalian host cell culture systems include established cell lines such as COS cells, L cells, 3T3 cells, Chinese hamster ovary (CHO) cells, embryonic stem cells, with HeLa cells being preferred.

The term “indicator gene” or “reporter gene” generically refers to an expressible (e.g., able to transcribed and (optionally) translated) DNA sequence which is expressed in response to the activity of a transcriptional regulatory protein. Indicator genes include unmodified endogenous genes of the host cell, modified endogenous genes, or a reporter gene of a heterologous construct, e.g., as part of a reporter gene construct. In a preferred embodiment, the level of expression of an indicator gene produces a detectable signal.

Reporter gene constructs are prepared by operatively linking an indicator gene with at least one transcriptional regulatory element. If only one transcriptional regulatory element is included it must be a regulatable promoter. In a preferred embodiment at least one of the selected transcriptional regulatory elements is indirectly or directly regulated by the activity of a transcriptional regulatory protein of the present invention, whereby activity of the transcriptional regulatory protein can be monitored via transcription of the reporter genes.

Many reporter genes and transcriptional regulatory elements are known to those of skill in the art and others may be identified or synthesized by methods known to those of skill in the art. Reporter genes include any gene that expresses a detectable gene product, which may be RNA or protein. Preferred reporter genes are those that are readily detectable. In one embodiment an indicator gene of the present invention is comprised in the nucleic acid molecule in the form of a fusion gene with a polynucleotide that includes desired transcriptional regulatory sequences.

Examples of reporter genes include, but are not limited to CAT (chloramphenicol acetyl transferase) (Alton and Vapnek (1979), Nature 282: 864-869) luciferase, and other enzyme detection systems, such as beta-galactosidase; firefly luciferase (deWet et al. (1987), Mol. Cell. Biol. 7:725-737); bacterial luciferase (Engebrecht and Silverman (1984), PNAS 1: 4154-4158; Baldwin et al. (1984), Biochemistry 23: 3663-3667); alkaline phosphatase (Toh et al. (1989) Eur. J. Biochem. 182: 231-238, Hall et al. (1983) J. Mol. Appl. Gen. 2: 101), human placental secreted alkaline phosphatase (Cullen and Malim (1992) Methods in Enzymol. 216:362-368), and horseradish peroxidase. In a preferred embodiment, the indicator gene is green fluorescent protein (U.S. Pat. No. 5,491,084; WO96/23898).

The term “detecting a signal produced by an indicator gene” is intended to include the detection of alterations in gene transcription of an indicator or reporter gene induced upon alterations in the activity of a transcriptional regulatory protein. In certain embodiments, the reporter gene may provide a selection method such that cells in which the transcriptional regulatory protein activates transcription have a growth advantage. For example the reporter could enhance cell viability, relieve a cell nutritional requirement, and/or provide resistance to a drug. In other preferred embodiments, the detection of an alteration in a signal produced by an indicator gene encompass assaying general, global changes to the cell such as changes in second messenger generation.

The amount of transcription from the reporter gene may be measured using any method known to those of skill in the art. For example, specific mRNA expression may be detected using Northern blots or specific protein product may be identified by a characteristic stain or an intrinsic activity. In preferred embodiments, the gene product of the reporter is detected by an intrinsic activity associated with that product. For instance, the reporter gene may encode a gene product that, by enzymatic activity, gives rise to a detection signal based on color, fluorescence, or luminescence.

The amount of activation of the indicator gene, e.g., expression of a reporter gene, is then compared to the amount of expression in a control cell. Control cells include cells that are substantially identical to the recombinant cells, but do not express one or more of the proteins encoded by the heterologous DNA, e.g., do not include or express a reporter gene construct, transcriptional regulatory protein, or selectable marker gene. Similarly, the amount of transcription of an indicator gene may be compared between a cell in the absence of a test modulator molecule and an identical cell in the presence of a test modulator molecule.

A “minimal activation domain” as used herein is intended to include a polypeptide sequence or fragment that comprises the transactivation potential of a transcriptional regulatory protein. A polypeptide encoding a minimal activation domain can be a naturally occurring polypeptide, e.g., it can be found within a protein that exists in nature, or it can be a polypeptide that has a composition that does not exist within a naturally occurring protein. In the context of the present invention a minimal activation domain is sufficient to confer upon a heterologous protein the ability to activate gene transcription. In a preferred embodiment, a minimal activation domain is derived from a 12 amino acid segment, residues 436 to 447, comprising the “acidic activation domain” of VP16.

The term “modulator”, as in “modulator of the transcription of a gene of interest” and “modulator of a transcriptional regulatory protein” is intended to encompass, in its various grammatical forms, induction and/or potentiation, as well as inhibition and/or downregulation of gene transcription and/or the activity of a transcriptional regulatory protein. In one embodiment, a method of the present invention encompasses the modulation of the transcription of an indicator gene in response to the activity of a transcriptional regulatory protein. In another embodiment, a method of the present invention encompasses the modulation of the activity of a transcriptional regulatory protein by a test compound which then results in a change in the transcription of a gene, preferably an indicator gene.

The term “operatively linked” or “operably linked” is intended to mean that molecules are functionally coupled to each other in that the change of activity or state of one molecule is affected by the activity or state of the other molecule. Nucleotide sequences are “operably linked” when the regulatory sequence functionally relates to the DNA sequence encoding the polypeptide or protein of interest. For example, a promoter nucleotide sequence is operably linked to a DNA sequence encoding the protein or polypeptide of interest if the promoter nucleotide sequence controls the transcription of the DNA sequence encoding the protein of interest. Typically, two polypeptides that are operatively linked are covalently attached through peptide bonds.

The term “selectable marker gene” or “selectable marker” is intended to include genes that encode a protein product that confers upon the cell expressing the protein product a phenotype that is distinguishable from cells that are not expressing the selectable marker gene. Selectable marker genes of the present invention include genes that confer amino acid or nucleotide prototrophy, antibiotic resistance, and metabolic drug resistance.

In the case of yeast, exemplary positively selectable (beneficial) genes include the following: URA3, LYS2, HIS3, LEU2, TRP1; ADE1,2,3,4,5,7,8; ARG1, 3, 4, 5, 6, 8; HIS1, 4, 5; ILV1, 2, 5; THR1, 4; TRP2, 3, 4, 5; LEU1, 4; MET2,3,4,8,9,14,16,19; URA1,2,4,5,10; H0M3,6,; ASP3; CHO1; ARO 2,7; CYS3; OLE1; IN01,2,4; PR01,3. Countless other genes are potential selectable markers. The above genes are involved in well-characterized biosynthetic pathways. In a preferred embodiment, a selectable marker gene is URA3 which encodes orotidine-5′-phosphate decarboxylase. URA3 expression can be used to confer growth in the absence of uracil. Conversely, URA3 is also a counterselectable or negatively selectable gene; loss of URA3 expression confers resistance to 5-fluoroorotic acid.

A “tetracycline analogue” is any one of a number of compounds that are closely related to tetracycline (Tc) and which bind to the tet repressor with a Ka of at least about 10⁶ M⁻¹. Preferably, the tetracycline analogue binds with an affinity of about 10⁹ M⁻¹ or greater, e.g., 10⁹M⁻¹. Examples of such tetracycline analogues include, but are not limited to those disclosed by Hlavka and Boothe, “The Tetracyclines,” in Handbook of Experimental Pharmacology 78, R. K.. Blackwood et al. (eds.), SpringerVerlag, Berlin-New York, 1985; L. A. Mitscher “The Chemistry of the Tetracycline Antibiotics, Medicinal Research 9, Dekker, New York, 1978; Noyee Development Corporation, “Tetracycline Manufacturing Processes,” Chemical Process Reviews, Park Ridge, N.J., 2 volumes, 1969; R. C. Evans, “The Technology of the Tetracyclines,” Biochemical Reference Series 1, Quadrangle Press, New York, 1968; and H. F. Dowling, “Tetracycline,” Antibiotics Monographs, no. 3, Medical Encyclopedia, New York, 1955; the contents of each of which are fully incorporated by reference herein. Examples of tetracycline analogues include anhydrotetracycline, doxycycline, chlorotetracycline, epioxytetracycline, cyanotetracycline and the like. Certain Tc analogues, such as anhydrotetracycline and epioxytetracycline, have reduced antibiotic activity compared to Tc.

The term “transcriptional regulatory domain” is intended to include the discrete domain of a transcriptional regulatory protein that modulates transcription of a gene. The mechanism by which a transcriptional regulatory domain modulates transcription includes, but is not limited to, direct or indirect interaction with elements of the basal transcription complex, e.g., RNA polymerase and TATA binding protein, direct or indirect interaction with other transcriptional regulatory proteins, and alteration of the conformation of the gene regulatory sequences. A transcriptional regulatory domain can either activate or inhibit transcription.

The Herpes simplex virion protein 16 contains two distinct transcriptional activation domains characterized by bulky, hydrophobic amino acids positioned in a highly negatively charged surrounding (Regier, J. L., Shen, F., and Triezenberg, S. J. (1993) Proc. Natl. Acad. Sci. U.S.A. 90, 883-887). Each domain was shown to activate transcription when fused to a heterologous DNA binding domain, such as the one of GAL4 (Seipel, K., Georgiev, O., and Schaffner, W. (1992) EMBO-J 11, 4961-4968).

In one embodiment, a transcriptional regulatory domain of the present invention is a polypeptide derived from the Herpes simplex virion protein 16. In another embodiment, a transcriptional regulatory domain includes at least one copy of a minimal activation domain of Herpes simplex virion protein 16. In a preferred embodiment, a transcriptional regulatory domain comprises an acidic region comprising amino acid residues 436 to 447 of the Herpes simplex virion protein 16.

The terms “transcriptional regulatory protein” and “transcriptional regulator” are used interchangeably and are intended to include any protein that is capable of modulating the transcription of a gene by contact, either directly or indirectly, with the gene regulatory sequences of the gene. Typically, the DNA binding and transcriptional activation or repression functions of a transcriptional regulatory protein, or transcription factor, are contained within discrete, modular domains of the protein. A transcriptional regulatory protein of the present invention includes a fusion protein comprising a polypeptide comprising a DNA binding protein operatively linked, e.g., functionally coupled, to a polypeptide comprising amino acid sequences derived from a transcriptional regulatory domain.

The term “variant allele” or “sequence variant” is intended to include a polynucleotide encoding a polypeptide or protein that comprises at least one mutation relative to the wild type allele. A mutation in a polynucleotide sequence is transferred to a mutation in the amino acid sequence encoded by said polynucleotide, and may thus affect protein structure and function. Types of mutations include silent, missense and nonsense mutations, as well as insertion and deletion mutations.

The present invention discloses a method for identifying transcriptional regulatory proteins that modulate the transcription of a gene of interest. As described herein, the method of the invention comprises introducing into a cell a nucleic acid molecule comprising three central components: 1) a polynucleotide (e.g., DNA) encoding a transcriptional regulatory protein; 2) an indicator gene which is responsive to, e.g., under the transcriptional control of, the gene regulatory sequences of a gene of interest which bind to the transcriptional regulatory protein; and 3) a selectable marker gene. The transcriptional regulatory protein binds to the gene regulatory sequences and either activates or inhibits transcription. A protein is identified as a modulator of the transcription of the gene of interest by detecting a signal generated by the indicator gene.

In one embodiment of the instant method, a cell is further treated with a modulator molecule, or analog thereof, that controls the binding of the transcriptional regulatory protein to the gene regulatory sequences. In a preferred embodiment, the binding of a transcriptional regulatory protein is dependent on the presence of a modulator molecule, or analog thereof. In a further preferred embodiment, the binding of a transcriptional regulatory protein is inhibited in the presence of a modulator molecule, or analog thereof.

In one embodiment, a transcriptional regulatory protein of the present invention is a tet repressor-based regulatory protein. A “tet repressor-based regulatory protein” is intended to include fusion proteins of the present invention that comprise a DNA binding domain derived from a tet repressor protein. A “tet repressor” protein or “TetR” refers to a prokaryotic protein which binds to a tet operator sequence in the absence but not the presence of tetracycline. The term “tet repressor” is intended to include repressors of different class types, e.g., class A, B, C, D or E tet repressors. A tet repressor-based regulatory protein comprises a polypeptide derived from TetR operatively linked to a polypeptide comprising a transcriptional regulatory domain. In a preferred embodiment, a transcriptional regulatory protein of the present invention is a tetracycline controlled transactivator protein. “Tetracycline controlled transactivators” or “tTA” are fusions between TetR and proper domains of transcriptional activators. The chimeric “tetracycline controlled transactivators” (tTA) allow one to regulate the expression of genes placed downstream of minimal promoter tetO fusions (Pₜₑₜ). In absence of Tc, Pₜₑₜ is activated whereas in presence of the antibiotic activation of Pₜₑₜ is prevented. In one embodiment, a polynucleotide encoding a polypeptide derived from the Herpes simplex virus protein 16 (VP16) is fused at the level of DNA to TetR. In another embodiment, a polynucleotide encoding at least one copy of a minimal activation domain of Herpes simplex VP16 is operably linked to TetR. In a further embodiment, a polynucleotide encoding at least one copy of an acidic region comprising amino acid residues 436 to 447 of Herpes simplex VP16 is operably linked to TetR.

In a further preferred embodiment, a transcriptional regulatory protein of the present invention is a reverse tetracycline controlled transactivator protein. A “reverse tetracycline controlled transactivator” or “rtTA” is a fusion protein comprising a TetR mutant which binds operator DNA only in presence of some tetracycline derivatives, or analogues, such as doxycycline (Dox) or anhydrotetracycline (ATc), operatively linked to a transcription activation domain. Thus, a rtTA protein will activate gene expression driven by Pₜₑₜ upon addition of Dox (Gossen et al., 1995). In one embodiment, a transcription activation domain of a rtTA protein is a polypeptide derived from the Herpes simplex virus protein 16 (VP16). In another embodiment, a transcription activation domain of a rtTA protein comprises at least one copy of an minimal activation domain of Herpes simplex VP16 is operably linked to TetR. In further embodiment, a transcription activation domain of a rtTA protein comprises at least one copy of an acidic region comprising amino acid residues 436 to 447 of Herpes simplex VP16 is operably linked to TetR.

In a preferred embodiment, a transcriptional regulatory protein of the present invention comprises a variant allele encoding a DNA binding protein operatively linked to a transcriptional regulatory domain. In one preferred embodiment, a mutation in a transcriptional regulatory protein confers a novel phenotype upon said protein. For example, a sequence variant of a transcriptional regulatory protein may have an altered DNA binding specificity, an altered transactivation potential, or an altered sensitivity to a modulator compound. In a preferred embodiment, a sequence variant of a transcriptional regulatory protein of the present invention comprises a mutant tTA protein. In another preferred embodiment, a sequence variant of a transcriptional regulatory protein of the present invention comprises a mutant rtTA protein.

There are numerous recognized ways to solve the structure-function problems of the sort presented by attempts to define the determinants involved in mediating the DNA binding and transcriptional regulatory function of a transcriptional regulatory protein. In one aspect of the present invention, specific mutations or alterations are introduced into a transcriptional regulatory protein based upon the available experimental evidence. In a second approach, random mutagenesis techniques, coupled with selection or screening systems, are used to introduce large numbers of mutations into a transcriptional regulatory protein, and that collection of randomly mutated proteins is then subjected to a selection for the desired phenotype or a screen in which the desired phenotype can be observed against a background of undesirable phenotypes.

With random mutagenesis one can mutagenize an entire molecule or one can proceed by cassette mutagenesis. In the former instance, the entire coding region of a molecule is mutagenized by one of several methods (chemical, PCR, doped oligonucleotide synthesis) and that collection of randomly mutated molecules is subjected to selection or screening procedures. Random mutagenesis can be applied in this way in cases where the molecule being studied is relatively small and there are powerful and stringent selections or screens available to discriminate between the different classes of mutant phenotypes that will inevitably arise. In the second approach, discrete regions of a protein, corresponding either to defined structural (i.e. α-helices, β-sheets, turns, surface loops) or functional determinants (e.g., DNA binding determinants, transcription regulatory domains) are subjected to saturating or semi-random mutagenesis and these mutagenized cassettes are re-introduced into the context of the otherwise wild type allele. Cassette mutagenesis is most useful when there is experimental evidence available to suggest a particular function for a region of a molecule and there is a powerful selection and/or screening approach available to discriminate between interesting and uninteresting mutants. Cassette mutagenesis is also useful when the parent molecule is comparatively large and the desire is to map the functional domains of a molecule by mutagenizing the molecule in a step-wise fashion, i.e. mutating one linear cassette of residues at a time and then assaying for function.

Random mutagenesis may be accomplished by many means, including:

1. PCR mutagenesis, in which the error prone Taq polymerase is exploited to generate mutant alleles of transcriptional regulatory proteins, which are assayed directly in yeast for an ability to couple. 2. Chemical mutagenesis, in which expression cassettes encoding transcriptional regulatory proteins are exposed to mutagens and the protein products of the mutant sequences are assayed directly in yeast for an ability to couple.

3. Doped synthesis of oligonucleotides encoding portions of the transcriptional regulatory protein gene.

4. In vivo mutagenesis, in which random mutations are introduced into the coding region of transcriptional regulatory proteins by passage through a mutator strain of E. coli, XL1-Red (mutD5 mutS mutT) (Stratagene, Menasa, Wis.). Substitution of mutant peptide sequences for functional domains in a transcriptional regulatory protein permits the determination of specific sequence requirements for the accomplishment of function.

The present invention also discloses a method for identifying a compound that modulates the activity of a transcriptional regulatory protein of interest. As described herein, the method of the invention comprises introducing into a cell a nucleic acid molecule comprising three central components: 1) a polynucleotide (e.g., DNA) encoding a transcriptional regulatory protein; 2) an indicator gene which is responsive to, e.g., under the transcriptional control of, the gene regulatory sequences of a gene of interest which bind to the transcriptional regulatory protein; and 3) a selectable marker gene. The transcriptional regulatory protein binds to the gene regulatory sequences, whereby the binding is controlled by a modulator compound, and activates gene transcription. The cell is treated with a test compound, and a compound is identified as a modulator of the transcriptional regulatory protein by detecting a signal generated by the indicator gene.

In one embodiment, a transcriptional regulatory protein binds to the gene regulatory sequences in the presence of a modulator compound. In a preferred embodiment, a transcriptional regulatory protein is a rtTA protein, or a sequence variant thereof. In another embodiment, the binding of a transcriptional regulatory protein to the gene regulatory sequences is inhibited in the presence of a modulator compound. In a further preferred embodiment, a transcriptional regulatory protein is a tTA protein, or a sequence variant thereof.

In certain embodiments of the instant invention, the compounds tested are in the form of peptides from a peptide library. The peptide library of the present invention takes the form of a cell culture, in which essentially each cell expresses one, and usually only one, peptide of the library. While the diversity of the library is maximized if each cell produces a peptide of a different sequence, it is usually prudent to construct the library so there is some redundancy. Depending on size, the combinatorial peptides of the library can be expressed as is, or can be incorporated into larger fusion proteins. The fusion protein can provide, for example, stability against degradation or denaturation. In an exemplary embodiment of a library for intracellular expression, e.g., for use in conjunction with intracellular target receptors, the polypeptide library is expressed as thioredoxin fusion proteins (see, for example, U.S. Pat. Nos. 5,270,181 and 5,292,646; and PCT publication WO94/02502). The combinatorial peptide can be attached on the terminus of the thioredoxin protein, or, for short peptide libraries, inserted into the so-called active loop.

In one embodiment, the test compound is exogenously added. In such embodiments the reagent cell is treated with the test compound. Exemplary compounds which can be screened for activity include, but are not limited to, peptides, nucleic acids, carbohydrates, small organic molecules, and natural product extract libraries. In accordance with the invention, both compounds which stimulate or inhibit the activity of the transcriptional regulatory protein can be selected and identified.

In another embodiment, the invention involves the use of a mixture of recombinant yeast cells to sample test compounds for identifying modulators of a transcriptional regulatory protein of interest. The ability of compounds to modulate the transcriptional activity of the target transcriptional regulatory protein can be scored for by detecting up or down-regulation of the detection signal provided by the indicator gene. Any difference, e.g., a statistically significant difference, in the amount of transcription indicates that the test compound has in some manner altered the activity of the specific transcriptional regulatory protein. After identifying certain test compounds as potential modulators of a transcriptional regulatory protein, the practitioner of the subject assay will continue to test the efficacy and specificity of the selected compounds both in vitro and in vivo. Whether for subsequent in vivo testing, or for administration to an animal as an approved drug, agents identified in the subject assay can be formulated in pharmaceutical preparations well known to the skilled artisan for in vivo administration to an animal, preferably a human.

The present invention further provides a method for identifying a gene regulatory sequence that binds to a transcriptional regulatory protein of interest. As described herein, the method of the invention comprises introducing into a cell a nucleic acid molecule comprising three central components: 1) a polynucleotide (e.g., DNA) encoding a transcriptional regulatory protein; 2) an indicator gene which is responsive to, e.g., under the transcriptional control of, the gene regulatory sequences of a gene of interest; and 3) a selectable marker gene. The transcriptional regulatory protein binds to the gene regulatory sequences of the gene of interest, and either activates or inhibits gene transcription. A gene regulatory sequence is identified as binding the transcriptional regulatory protein by detecting a signal generated by the indicator gene.

In one embodiment of the instant method, a cell is further treated with a modulator molecule, or analog thereof, that controls the binding of the transcriptional regulatory protein to the gene regulatory sequences. In a preferred embodiment, the binding of a transcriptional regulatory protein is dependent on the presence of a modulator molecule, or analog thereof. In a further preferred embodiment, the binding of a transcriptional regulatory protein is inhibited in the presence of a modulator molecule, or analog thereof.

In one embodiment, the gene regulatory sequences comprise variants of the gene regulatory sequences of the gene of interest having at least one nucleotide substitution. A nucleotide substitution within the DNA binding site of a gene regulatory sequence may influence the affinity of the cognate DNA binding protein for its binding site. In a preferred embodiment, the gene regulatory sequences comprise sequences derived from the Tet operator which have at least one nucleotide substitution.

In one embodiment, a transcriptional regulatory protein is a Tet repressor-based regulatory protein that binds to regulatory sequences derived from the Tet operator. In a preferred embodiment, a transcriptional regulatory protein is a rtTA protein, or a sequence variant thereof. In a further preferred embodiment, a transcriptional regulatory protein is a tTA protein, or a sequence variant thereof.

The present invention also discloses recombinant vector constructs and recombinant host cells transformed with said constructs. The term “recombinant vector” is intended to include any plasmid, phage DNA, or other DNA sequence which is able to replicate autonomously in a host cell. A vector may be characterized by one or a small number of restriction endonuclease sites at which such DNA sequences may be cut in a determinable fashion without the loss of an essential biological function of the vector, and into which a DNA fragment may be spliced in order to bring about its replication and cloning. A vector may further contain a marker suitable for use in the identification of cells transformed with the vector. Recombinant vectors may be generated to enhance the expression of a gene which has been cloned into it, after transformation into a host. The cloned gene is usually placed under the control of (i.e., operably linked to) certain control sequences or regulatory sequences, which may be either constitutive or inducible.

A recombinant expression vector of the invention can be a virus, or portion thereof, which allows for expression of a nucleic acid introduced into the viral nucleic acid. For example, replication defective retroviruses, adenoviruses and adeno-associated viruses can be used. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are well known to those skilled in the art. Examples of suitable packaging virus lines include ψCrip, ψCre, ψ2 and ψAm. The genome of adenovirus can be manipulated such that it encodes and expresses a transcriptional regulatory protein but is inactivated in terms of its ability to replicate in a normal lytic viral life cycle. See for example Berkner et al. (1988) BioTechniques 6:616; Rosenfeld et al. (1991) Science 252:431-434; and Rosenfeld et al. (1992) Cell 68:143-155. Suitable adenoviral vectors derived from the adenovirus strain Ad type 5 d1324 or other strains of adenovirus (e.g., Ad2, Ad3, Ad7 etc.) are well known to those skilled in the art. Alternatively, an adeno-associated virus vector such as that described in Tratschin et al. (1985) Mol. Cell. Biol. 5:3251-3260 can be used to express a transcriptional regulatory protein of the present invention.

In general, it will be desirable that an expression vector be capable of replication in the host cell. Heterologous DNA may be integrated into the host genome, and thereafter is replicated as a part of the chromosomal DNA, or it may be DNA which replicates autonomously, as in the case of a plasmid. In the latter case, the vector will include an origin of replication which is functional in the host. In the case of an integrating vector, the vector may include sequences which facilitate integration, e.g., sequences homologous to host sequences, or encoding integrases.

Appropriate cloning and expression vectors for use with bacterial, fungal, yeast, and mammalian cellular hosts are known in the art, and are described in, for example, Powels et al. (Cloning Vectors: A Laboratory Manual, Elsevier, N.Y., 1985). Mammalian expression vectors may comprise non-transcribed elements such as an origin of replication, a suitable promoter and enhancer linked to the gene to be expressed, and other 5′ or 3′ flanking nontranscribed sequences, and 5′ or 3′ nontranslated sequences, such as necessary ribosome binding sites, a poly-adenylation site, splice donor and acceptor sites, and transcriptional termination sequences.

A number of vectors exist for the expression of recombinant proteins in yeast. For instance, YEP24, YIP5, YEP51, YEP52, pYES2, and YRP17 are cloning and expression vehicles useful in the introduction of genetic constructs into S. cerevisiae (see, for example, Broach et al. (1983) in Experimental Manipulation of Gene Expression, ed. M. Inouye Academic Press, p. 83, incorporated by reference herein). These vectors can replicate in E. coli due to the presence of the pBR322 ori, and in S. cerevisiae due to the replication determinant of the yeast 2 micron plasmid. In addition, drug resistance markers, e.g., antibiotics which confer resistance in fungal systems, can be used. Suitable promoters for function in yeast include the promoters for metallothionein, 3-phosphoglycerate kinase (Hitzeman et al., J. Biol. Chem. 255, 2073 (1980) or other glycolytic enzymes (Hess et al., J. Adv. Enzyme Req. 7, 149 (1968); and Holland et al. Biochemistry 17, 4900 (1978)), such as enolase, glyceraldehyde-3-phosphate dehydrogenase, hexokinase, pyruvate decarboxylase, phospho-fructokinase, glucose-6-phosphate isomerase, 3-phosphoglycerate mutase, pyruvate kinase, triosephosphate isomerase, phospho-glucose isomerase, and glucokinase. Suitable vectors and promoters for use in yeast expression are further described in R. Hitzeman et al., EPO Publication. No. 73,657. Other promoters, which have the additional advantage of transcription controlled by growth conditions, are the promoter regions for alcohol dehydrogenase 2, isocytochrome C, acid phosphatase, degradative enzymes associated with nitrogen metabolism, and the aforementioned metallothionein and glyceraldehyde-3-phosphate dehydrogenase, as well as enzymes responsible for maltose and galactose utilization. Finally, promoters that are active in only one of the two haploid mating types may be appropriate in certain circumstances. Among these haploid-specific promoters, the pheromone promoters MFa1 and MFα1 are of particular interest.

In one embodiment, a recombinant vector comprises a nucleic acid molecule comprising polynucleotide elements encoding 1) a fusion protein that regulates transcription; 2) an indicator gene, the expression of which is responsive to said fusion protein; and 3) a selectable marker gene. The fusion protein comprises a first polypeptide derived from a DNA binding domain of a protein that binds to a regulatory sequence of a gene of interest in operative linkage with a second polypeptide derived from a transcriptional regulatory domain.

In another embodiment, a recombinant vector comprises a nucleic acid molecule comprising polynucleotide elements encoding 1) a Tet repressor-based regulatory protein; 2) an indicator gene, the expression of which is responsive to said Tet repressor-based regulatory protein; and 3) a selectable marker gene. The Tet repressor-based regulatory protein comprises a first polypeptide derived from a Tet repressor protein that binds to a regulatory sequence of a gene of interest in operative linkage with a second polypeptide derived from a transcriptional regulatory domain. The binding of the Tet repressor-based regulatory protein to the gene regulatory sequence is controlled by a modulator molecule, or an analog thereof.

In a preferred embodiment, a recombinant vector comprises polynucleotide elements encoding 1) a Tet repressor-based regulatory protein, 2) an indicator gene, the expression of which is regulated by sequences derived from the Tet operator; and 3) a selectable marker gene. The Tet repressor-based regulatory protein comprises a first polypeptide derived from a Tet repressor protein that binds to regulatory sequences derived from the Tet operator in operative linkage to a second polypeptide derived from a transcription activation domain. The binding of the Tet repressor-based regulatory protein to the sequences derived from the Tet operator is controlled by tetracycline, or an analog thereof.

The present invention is widely applicable to a variety of situations where it is desirable to be able to turn gene expression on and off, or regulate the level of gene expression, in a rapid, efficient and controlled manner without causing pleiotropic effects or cytotoxicity. For example, the proteins, modulator compounds and gene regulatory sequences identified by the methods of the invention have use in the study of cellular development and differentiation in eukaryotic cells, plants and animals. The expression of oncogenes can be regulated in a controlled manner in cells to study their function. Additionally, the system can be used to regulate the expression of site-specific recombinases, such as CRE or FLP, to thereby allow for irreversible modification of the genotype of a transgenic organism under controlled conditions at a particular stage of development. For example, drug resistance markers inserted into the genome of transgenic plants that allow for selection of a particular transgenic plant could be irreversibly removed via a Tc-regulated site specific recombinase.

The present invention may also be particularly useful for gene therapy purposes, in treatments for either genetic or acquired diseases. The general approach of gene therapy involves the introduction of nucleic acid into cells such that one or more gene products encoded by the introduced genetic material are produced in the cells to restore or enhance a functional activity. For reviews on gene therapy approaches see Anderson, W. F. (1992) Science 256:808-813; Miller, A. D. (1992) Nature 357:455-460; Friedmann, T. (1989) Science 244:1275-1281; and Cournoyer, D., et al. (1990) Curr. Opin. Biotech. 1:196-208. However, current gene therapy vectors typically utilize constitutive regulatory elements which are responsive to endogenous transcriptions factors. These vector systems do not allow for the ability to modulate the level of gene expression in a subject. In contrast, the proteins, modulator compounds and gene regulatory sequences identified by the methods of the invention provides the ability to modulate gene expression in a cell in vitro or in vivo.

### EXEMPLIFICATION

The following examples are provided to further illustrate various aspects of the present invention. They are not to be construed as limiting the invention.

The screen described in the following examples is based on the tTA/rtTA dependent expression of green fluorescent protein (GFP) from aequorae victoria (Niedenthal et al., 1996; Wach et al., 1998; Oldenburg et al., 1997, as optimized for enhanced fluorescence). The GFP protein was optimized for enhanced fluorescence by inserting the following mutations: F99S, M153T, and V163A (according to Crameri et al. (1996), Nature Biotechnology 14, 315-319); F64L and S65T (according to Cormack et al. (1996), Gene 173, 33-38); and Q80R and the insertion of an alanine at position 2; yielding GFP+. The fluorescence of GFP expressing yeast colonies is conveniently detected on suitable agar plates under UV light and can be quantified by FACS or fluorescence spectroscopy (Niedenthal et al., 1996).

Thus, a plasmid, designated pCM 190GFP+, was constructed which contains the following elements:
the coding sequence for GFP controlled by a tTA/rtTA responsive promoter;
a tTA/rtTA encoding sequence that is constitutively expressed;
the URA3 marker that allows selection in appropriate yeast strains;
the replication function of the 2μ episome of S.cerevisiae.
   Unique endonuclease cleavage sites allow for the removal of the TetR, the activation domain, or the entire tTA encoding sequences, typically replaced by pools of mutagenized alleles obtained as described previously (Leung et al., 1989).

The plasmid mixture was transformed to S. cerevisiae and transformants were selected via uracil prototrophy. The resulting transformants were screened on agar plates that allow examination for a variety of properties:
induction of GFP by Tc derivatives or other chemicals;
new alleles of rtTA with reduced basal expression in the absence of the inducer;
increased expression levels in the presence of inducers.

If the sequence encoding the activating domain is replaced by appropriate sequence libraries, the screen can identify new activator or silencer domains that function optimally in fusions with TetR variants.

### Example 1

### rtTA Variants with Improved Properties

The gene encoding GFP was cloned into the multiple cloning site of pCM190 (Gari et al., 1997) to serve as an indicator of rtTA activity, yielding plasmid pCM190GFP+. The TetR portion of tTA was amplified for mutagenesis with two oligonucleotide primers, 5′-GACCGATCCAGCCTCCGCGG (SEQ ID NO:1), and 5′-CGTGTGTCCCGCGGGGAGAA (SEQ ID NO:2), from the vector pCM190 as described (Leung et al., 1989). The PCR-fragments and pCM190-GFP+ were restricted with XbalI and BsiWI and purified. The PCR-fragments and the vector were then ligated and transformed into E. coli DH5α. Several thousand E. coli clones were co-cultured, and their plasmid pools were prepared and transformed into S. cerevisiae using the LiAc-method (Ito et al., 1983). The RS453 strain of S. cerevisiae (MATa; ade2-1; trp1-1; can1-100; leu2-3,112; his3- 1; ura3-52) was used for the screening protocol.

Transformation of the plasmid into yeast allows one to score differences in GFP activity over a wide range of intensities by direct examination of colonies placed in UV light. In this way, large populations of yeast cells can be screened for promising tTA/rtTA candidates. Differences in the fluorescence of GFP originate from different expression levels of the indicator protein. This will, in general, reflect differences in the activation potential of the transactivators. After the usual screen, biochemical analysis can be performed with only a small number of positive candidates.

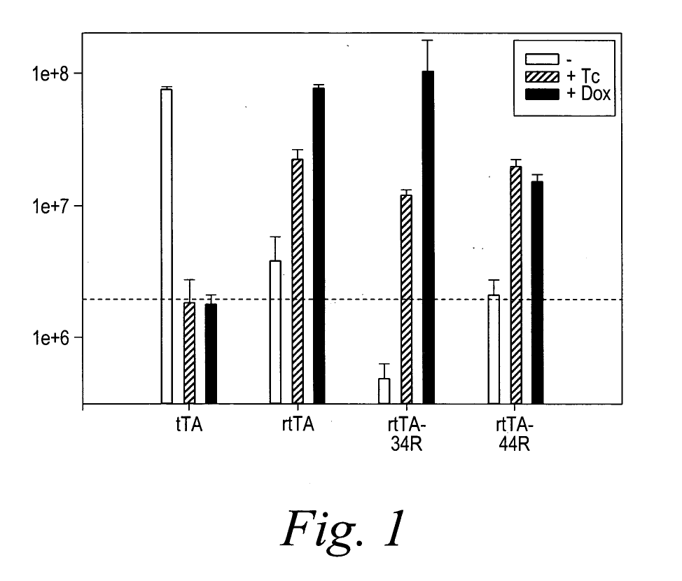

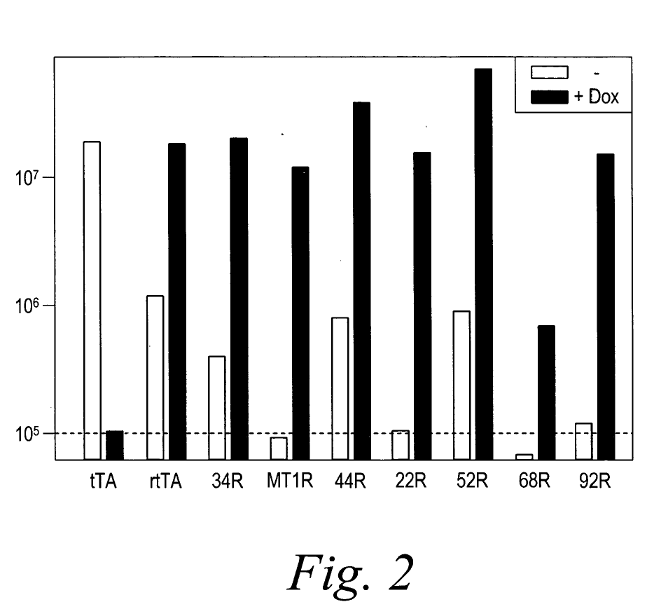

Accordingly, the resulting uracil-prototroph yeast clones were replica plated on minimal medium without uracil containing either Tc and/or Dox and scored after growth for two to three days at 30° C. using long wavelength UV-light to excite GFP fluorescence. This led to the identification of several new rtTA-alleles: 34R, 44R, MT1R, 22R, 52R, 68R and 92R. The phenoype of the rtTA-34R and rtTA-44R alleles in yeast stimulated with Tc and Dox are shown in FIG. 1. The phenotype of the 34R, 44R, MT1R, 22R, 52R, 68R and 92R alleles in yeast stimulated with Dox are shown in FIG. 2. The GFP fluorescence is shown on a logarithmic scale on the left axis. Fluorescence intensities are shown for each transactivator in the absence of inducer, in the presence of 10 μg/ml of Tc, and/or in the presence of 10 μg/ml of Dox. The activities achieved with tTA and rtTA are shown for comparison.

S. cerevisiae strains containing rtTA-34R, rtTA-44R and a GFP⁻ strain, as well as strains containing the original tTA and rtTA were grown overnight in minimal medium. Equivalents containing 1 OD600 of the cells were harvested, washed with PBS, and suspended in 2 ml of PBS. The light emission of these cells was scored in a fluorimeter using an excitation wavelength of 490 nm and recording emission at 512 nm. The basal activities of rtTA-34R and rtTA-44R were clearly lower as compared to rtTA. As shown in FIG. 1, activation of expression was at least in one case slightly higher than that achieved with the original rtTA or tTA, respectively. The induction factors varied between 100 and 300-fold. Thus, the new rtTA alleles are much better suited for regulation of gene expression in yeast than the original rtTA, which leads to only 40-fold induction of expression.

The advantage of the new rtTA's are low basal activities in the uninduced state combined with high levels of induction upon addition of Tc or Dox. This is achieved in absence of any repressor and thus permits regulation of gene expression over a broad range even in S. cerevisiae.

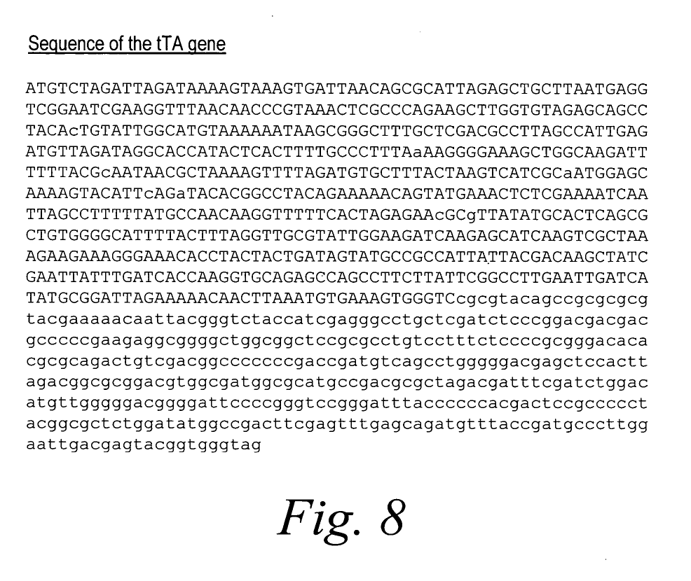

Following isolation of the respective plasmids from S. cerevisiae, the mutagenized rtTA regions were sequenced. The genotype of the novel rtTA alleles is shown in Table 1 below. The reference sequence of the parent rtTA is shown in FIG. 8.

TABLE 1
Novel rtTA mutants

| | 1^{st} aa | 2^{nd} aa | 3^{rd} aa | 4^{th} aa | 5^{th} aa | 6^{th} aa |
|---|---|---|---|---|---|---|
| Designation of rtTA | exchange/ | exchange/ | exchange/ | exchange/ | exchange/ | exchange/ |
| sequence variant | new codon | new codon | new codon | new codon | new codon | new codon |
| rtTA-34R | E19G | A56P | H139H | D148E | H179R | |
| | ggg | ccc | cac | gaa | cgc | |
| rtTA-1956R | E19G | A56P | | | | |
| | ggg | ccc | | | | |
| rtTA-MT1R | S12G | E19G | A56P | | | |
| | ggc | ggg | ccc | | | |
| rtTA-MT1/34R | S12G | E19G | A56P | H139H | D148E | H179R |
| | ggc | ggg | ccc | cac | gaa | cgc |
| rtTA-44R | T26A | D95G | | | | |
| | gca | ggt | | | | |
| rtTA-22R | G96R | | | | | |
| | aga | | | | | |
| rtTA-52R | V99E | | | | | |
| | gaa | | | | | |
| rtTA-68R | E19G | R87S | ΔC88 | | | |
| | ggg | agt | — | | | |
| rtTA-92R | V99E | E204K | | | | |
| | gaa | aaa | | | | |

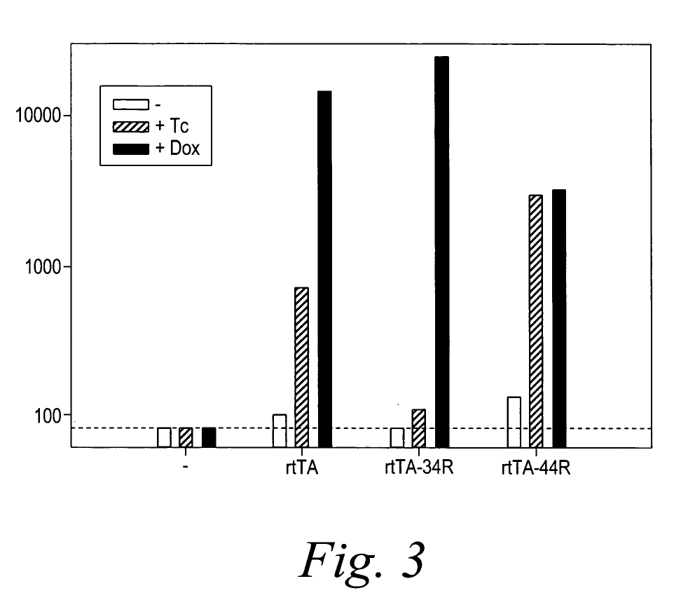

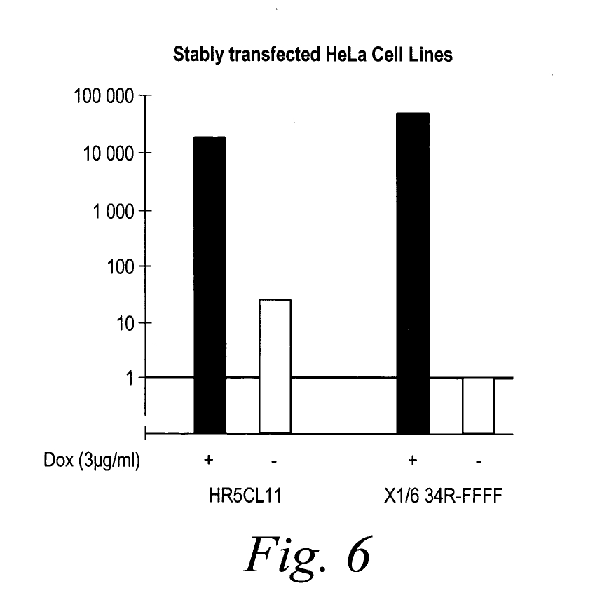

rtTA-34R and rtTA-44R were then recloned into pUHD15-1 (Gossen & Bujard, 1992), replacing the respective portions of tTA. HeLa cells were transiently cotransfected with plasmids pUHC13-3, encoding the luciferase gene controlled by Pₜₑₜ (Gossen & Bujard, 1992), and the pUHD15-1 plasmids containing the genes of the respective transactivators. Luciferase activities were measured in absence (light column) and presence of 5 μg/ml of the effectors tetracycline (Tc, light grey) or doxycycline (Dox, dark grey). On the X axis, (−) corresponds to control HeLa cells into which no DNA was transferred. The results shown in FIG. 3 indicate that rtTA-34R may lead to an even higher induction of luciferase activity as compared to rtTA. The increased regulation factor observed results from both a lower basal and a higher induced activity. Thus, rtTA-34R isolated exhibits an improved reverse phenotype in HeLa cells as well as in S. cerevisiae (FIGS. 3 and 6). As in S. cerevisiae, the mutant rtTA-44R also shows a reverse phenotype in HeLa cells. However, when compared with rtTA, the induction level is not improved over rtTA.

Thus, the described screening procedure for new rtTA alleles identifies mutants which show induction of transcription after Dox addition in HeLa cells. Furthermore, the phenotypes observed in HeLa cells for most mutants reflect faithfully the properties seen in yeast. This demonstrates that the screening procedure in S. cerevisiaie is a valuable tool for discovering TetR-based regulatory proteins with novel activities in mammalian cells.

### Example 2

### Selection of tTA Mutants with Differential Induction by Tetracycline Analogues

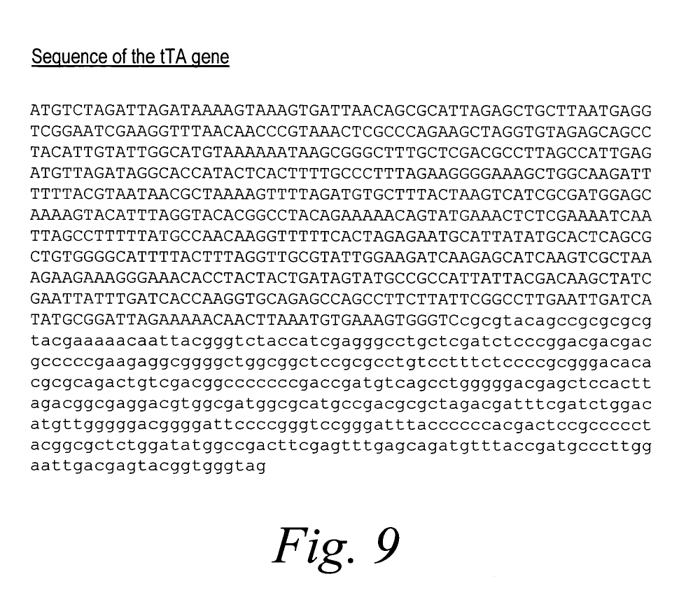

In order to identify tTA mutations with different sensitivities towards tetracycline analogues, mutagenesis of the TetR portion of tTA, transformation and selection in yeast were performed as outlined above. For further analysis, the resulting candidates were transformed into yeast and spread on minimal medium plates in the absence of uracil, which contained either 10 μg/ml tetracycline, anhydrotetracycline, oxy-tetracycline, chloro-tetracycline or doxycycline. The yeast were grown for two to three days at 30° C. and their GFP expression phenotype was examined as described above. This led to the identification of several new tTA-alleles: 2, 11, 19, 22, 23, 24, 31, 36, 38, 45, and 50; the genotype of the novel tTA alleles is shown in Table 2 below. The reference sequence of the parent tTA is shown in FIG. 9.

TABLE 2
Novel tTA mutants

| | 1^{st} aa | 2^{nd} aa | 3^{rd} aa | 4^{th} aa | 5^{th} aa | 6^{th} aa |
|---|---|---|---|---|---|---|
| Designation of tTA | exchange/ | exchange/ | exchange/ | exchange/ | exchange/ | exchange/ |
| sequence variant | new codon | new codon | new codon | new codon | new codon | new codon |
| tTA-2 | P167S | | | | | |
| | tcg | | | | | |
| tTA-11 | I164L | | | | | |
| | ctt | | | | | |
| tTA-19 | F78S | | | | | |
| | tct | | | | | |
| tTA-22 | Y132C | | | | | |
| | tgt | | | | | |
| tTA-23 | Y110C | I174V | | | | |
| | tgt | gtc | | | | |
| tTA-24 | I174T | E183K | | | | |
| | acc | aag | | | | |
| tTA-31 | L113H | | | | | |
| | cac | | | | | |
| tTA-36 | S85G | I174V | | | | |
| | ggt | gtc | | | | |
| tTA-38 | S85R | | | | | |
| | aga | | | | | |
| tTA-45 | D77D | L170V | L187L | | | |
| | gac | gta | ttg | | | |
| tTA-50 | A56V | | | | | |
| | gtc | | | | | |

One mutant that was isolated from this screen, tTA-45, was sequenced and found to carry an amino acid exchange at position 170 from a leucine to a valine (L170V). The induction efficiency of tTA-45 in response to varying concentrations of Tc or Dox was determined in transient transfection assays in HeLa cells. The inducer concentration leading to 50% repression of the luciferase activity (IC₅₀) was determined and is described in Table 3 below.

TABLE 3
Effects of Tc and Dox on induction properties of tTA and tTA-45

IC₅₀ (ng inducer/ml)

| Inducer | tTA (Wildtype) | tTA-45 (L170V) |
|---|---|---|
| Tc | 3 | 270 |
| Dox | 0.6 | 5 |

The mutant tTA-45 is 100-fold less sensitive to Tc, but only about 10-fold less sensitive to Dox.

Therefore, we conclude that the S. cerevisiae based screen for Tc dependent eukaryotic transcriptional activators is also suitable for the identification of tTAs with altered inducer recognition properties. This is important for practical applications because this screen can be used to change the induction profiles of Tc dependent transcription factors, thus enabling the construction of novel alleles which respond differentially to chemically distinct inducers. These Tc dependent transcription factors may then be used to construct mammalian cell lines or transgenic animals in which a number of different genes can be differentially regulated by various combinations of Tc analogues.

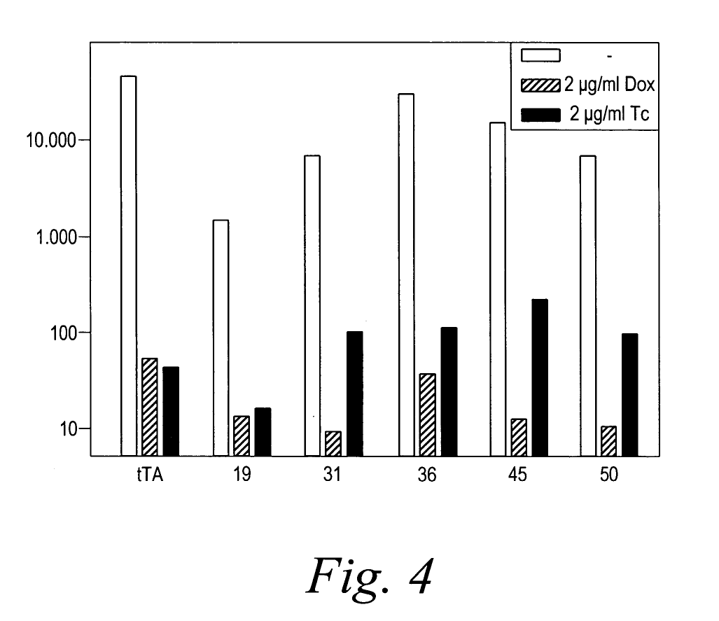

Analysis of five novel tTA alleles, tTA-19, tTA-31, tTA-36, tTA-45 and tTA-50, was performed by transient transfection into human epithelial cells. Luciferase activities were measured in absence (light column) and presence of 2 μg/ml of the effectors doxycycline (Dox, light grey) or tetracycline (Tc, dark grey), as shown in FIG. 4. On the X axis, (−) corresponds to control human epithelial cells into which no DNA was transferred.

### Example 3

### A Novel TetR-Based Transactivator: rtTA-34R

The new allele encoding the reverse Dox-inducible transactivator rtTA-34R was sequenced and found to contain different mutations than the previously characterized rtTA. This demonstrates that a reverse transactivator phenotype can be obtained by mutations in different regions of TetR. The mutations found in rtTA-34R are: E19G, A56P, H139H (silent), A148E, and H179R. The amino acids at positions 95, 101, and 102, which are mutated in the original rtTA are the wild type residues in rtTA-34R.

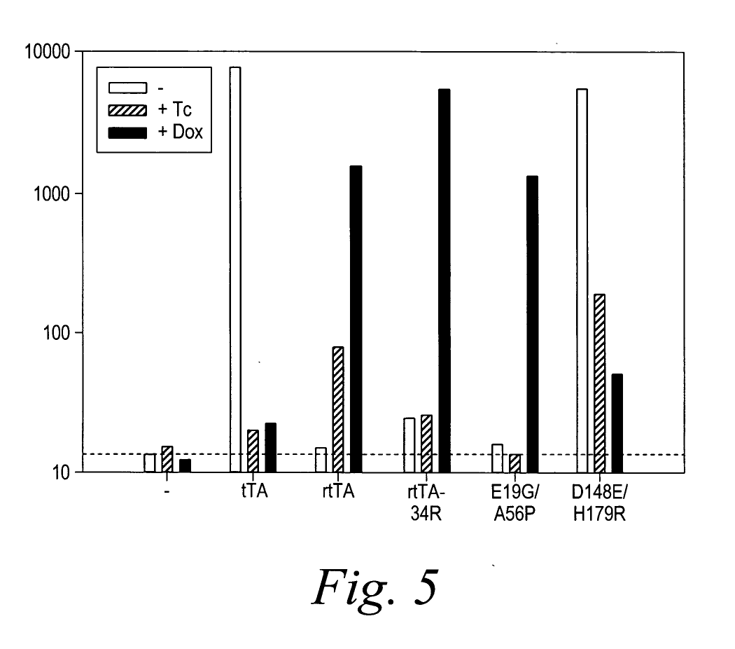

To obtain additional information about the role of the mutated residues, we separated the mutations at positions 19 and 56 from those at 139, 148 and 179. The resulting proteins are called rtTA-1956R and rtTA-148179R. The activation potential of rtTA-1956R and rtTA-148179R was assessed in transient expression experiments. Plasmids encoding the respective rtTA variants were cotransfected with the pUHC13-3 luciferase indicator plasmid into HeLa cells and the luciferase activity was determined. The results shown in FIG. 5 indicate that that two exchanges, E19G and A56P, are sufficient for the reverse phenotype. The mutations in positions 148 and 179 are merely slightly supportive for the phenotype as they do not yield a reverse phenotype by themselves.

### Example 4

### HeLa Cell Lines Producing rtTA-34R from Episomally Stabilized Plasmids

In order to generate cell lines that maintain the plasmid episomally and thus produce the transactivator over extended periods of time, the transcription unit containing the rtTA-34R coding sequence controlled by the hCMV promoter was inserted into pREP9 (Invitrogen, Carlsbad, USA) from which the RSV promoter had been excised. This resulted in the Epstein Barr-based vector pCEP4-rtTA-34R. HeLa cells were transfected with the plasmid pCEP4-rtTA-34R, and clones isolated via G418 selection. Clones stably producing the transactivator were selected and tested for their ability to activate transcription from the transiently transfected luciferase reporter construct pUHC13-3 in the presence and absence of Dox.

The data shown in Table 4 indicates that three HeLa cell lines derived from various clones (0.34R-16, -33 and -36) exhibit similar background activity slightly higher than the parent cell line. Upon addition of 5 μg/ml of Dox, luciferase activity is induced up to 600 fold. In comparison to the HeLa cell line HR5 harboring chromosomal copies of the rtTA gene (Gossen et al., 1995), the background level is reduced. In addition, the induced level of luciferase is significantly elevated. This leads to a several hundred fold induction of gene expression in the case of the rtTA-34R clones whereas in HR5 cells rtTA achieves only a 20 to 30 fold induction under these conditions.

TABLE 4
Doxycycline-dependent regulation of luciferase in HeLa cells
producing rtTA-34R from episomally stabilized plasmids.

| | Luciferase activity | | |
|---|---|---|---|
| | (RLU/μg protein) | | factor |
| Cell line | with Dox | without Dox | of induction |
| HeLa | 430 ± 110 | 324 ± 20 | 1 |
| HeLa HR5 (Tet on) | 920 ± 170 | 26400 ± 4030 | 27 |
| HeLa 0.34R-16 | 540 ± 50 | 323600 ± 69470 | 600 |
| HeLa 0.34R-33 | 360 ± 140 | 74620 ± 3230 | 200 |
| HeLa 0.34R-36 | 430 ± 50 | 87000 ± 7820 | 200 |

### Example 5

### Gene Encoding rTetR-34R Allele Fused to Minimal Activation Domains

The coding sequence of rTetR-34R was fused with a DNA encoding four minimal activation domains (FFFF)(Baron et al., 1997) by insertion into a proper pUHD vector to generate plasmid pUHrT61-1. HeLa cell line X1/6 was transfected with plasmid pUHrT51-1 carrying the rtTA-34R-FFFF gene under the control of P_{hCMV}. The resulting HeLa cell line X1/6-34R-FFFF contains, in addition, the Pₜₑₜ-luciferase expression unit in a “silent but activatable” locus.

Cell lines derived from various clones that contain pUHrT51-1 stably inserted into the genome where it is constitutively expressed were isolated via hygromycin-B selection and analyzed for Dox dependent luciferase activity. As shown in FIG. 6, in the absence of Dox there was no detectable luciferase activity, whereas upon addition of Dox, luciferase activity was induced up to 50 000 fold. In contrast, in our previously described HR5-CL11 cell line (Gossen et al., 1995), a significant background luciferase activity is observed in the absence of Dox and induction by Dox reaches only about 700 fold. This is most likely due to the residual affinity between rtTA and tetO.

### Example 6

### A synthetic Gene Encoding TetR-34R Fused to Minimal Activation Domains (rtTA2-34R^{S})

To further improve rtTA-34R, the DNA sequence encoding rtTA-34R fused to 3 minimal activation domains (FFF) was converted into a polynucleotide that encodes the transactivator in codon frequencies as found in humans. This rtTA2-34R^{S} sequence was optimized with respect to a variety of additional parameters as described previously (Pan et al., 1999). Thus, it contains neither splice donor nor splice acceptor sites. Other features that might limit its expression have been eliminated as well. It is anticipated that with this synthetic gene, rtTA2-34R^{S} can be stably produced in a variety of eukaryotic cells that are presently not amenable to rtTA-mediated gene regulation. This is currently being examined through the generation of several transgenic mouse lines that are expected to produce rtTA2-34R^{S} in hepatocytes and in mature B-cells.

The synthetic gene encoding rtTA2-34R was cloned into pUHD15-1 expression was examined in HeLa cells. In transient transfection experiments using luciferase activity in relative light units as a functional readout, induction of up to 20 fold was observed in cells treated with Dox, as shown in Table 5.

TABLE 5
Activity of rtTA2-34R^{s} in HeLa Cells

| | Experiment A | Experiment B |
|---|---|---|
| −Dox | 1 | 1 |
| +Dox | 20.325 | 7.279 |

Cellular material from cells transfected as described above was also used to compare the binding of rtTA2-34R^{S} and rtTA2 (rtTA fused to 3 minimal activation domains) to operator DNA in DNA retardation experiments.

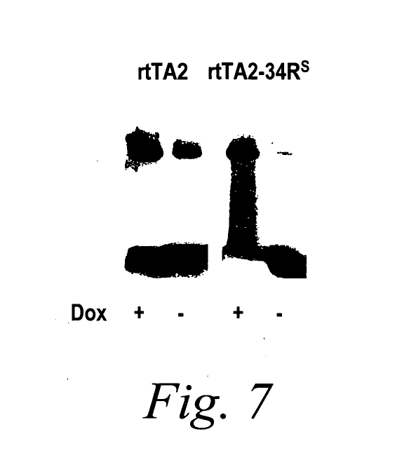

rtTA2 and rtTA2-34R^{S} were produced in HeLa cells and exposed to radioactively labeled tetO DNA in presence (+) and absence (−) of Dox. Electrophoretic migration of the complexes reveals the differential affinities between tetO and the two transactivators. As indicated in FIG. 7, the residual binding (i.e., binding in the absence of Dox) of rtTA2-34R^{S} to operator DNA is greatly reduced.

Therefore, the new reverse transactivator is a decisive improvement when compared to the previously characterized rtTA. Since there is little reason to assume that the screening performed for this result was saturating, we anticipate that other rtTA's with still improved properties may be obtained.

References:

Baron, U., Gossen, M. & Bujard, H. (1997) Tetracycline controlled transcription in eukaryotes: novel transactivators with graded transactivation potential. Nucl. Acids Res. 25, 2723-2729.
Baron, U., Schnappinger, D., Helbl, V., Gossen, M., Hillen, W. & Bujard, H. (1999) Generation of conditional mutants in higher eukaryotes by switching between the expression of two genes. Proc. Natl. Acad. Sci. USA 96, 1013-1018.
Belli, G., Gari, E., Piedrafita, L., Aldea, M., & Herrero, E. (1998). An activator/repressor dual system allows tight tetracycline-regulated gene expression in budding yeast. Nucleic Acids Res. 26, 942-947.
Bello, B., Resendez-Perez, D., & Gehring, W. J. (1998). Spatial and temporal targeting of gene expression in Drosophila by means of a tetracycline-dependent transactivator system. Development 125, 2193-2202.
Bieschke, E. T., Wheeler, J. C., & Tower, J. (1998). Doxycycline-induced transgene expression during Drosophila development and aging. Mol. Gen. Genet. 258, 571-579.
Camacho-Vanegas, O., Mannucci, L. & Amaldi, F. (1998) Construction of Xenopus (B3.2) and human (HeLa) cell lines expressing the tetracycline-controlled transactivator (tTA). In Vitro Cell Dev. Biol. Anim. 34, 14-15.
Cohlan, S. Q. (1977). Tetracycline staining of teeth. Teratology 15, 127-129.
Efrat, S., Fusco-DeMane, D., Lemberg, H., Emran, O. A. & Wang, X. (1995) Conditional transformation of a pancreatic β-cell line derived from transgenic mice expressing a tetracycline regulated oncogene. Proc. Natl. Acad. Sci. USA 92, 3576-3580.
Ewald, D., Li, M., Efrat, S., Auer, G., Wall, R. J., Furth, P. A. & Hennighausen, L. (1996) Time-sensitive reversal of hyperplasia in transgenic mice expressing SV40 T antigen. Science 273, 1384-1386.
Fishman, G. I., Kaplan, M. L. & Buttrick, P. M. (1994) Tetracycline-regulated cardiac gene expression in vivo. J. Clin. Invest. 93, 1864-1868.
Früh, K., Gossen, M., Wang, K., Bujard, H., Peterson, P. A., & Yang, Y. (1994). Displacement of housekeeping proteasome subunits by MHC-encoded LMPs: a newly discovered mechanism for modulating the multicatalytic proteinase complex. EMBO J. 13, 3236-3244.
Furth, P. A., Onge, L. S., Böger, H., Gruss, P., Gossen, M., Kistner, A., Bujard, H., & Henninghausen, L. (1994). Temporal control of gene expression in transgenic mice by a tetracycline-responsive promoter. Proc. Natl. Acad. Sci. USA 91, 9302-9306.
Gari, E., Piedrafita, L., Aldea, M., & Herrero, E. (1997). A set of vectors with a tetracycline-regulatable promoter system for modulated gene expression in Saccharomyces cerevisiae. Yeast 13, 837-848.
Gatz, C., & Quail, P. H. (1988). Tn10-encoded Tet repressor can regulate an operator-containing plant promoter. Proc. Natl. Acad. Sci. USA 85, 1394-1937.
Gossen, M., & Bujard, H. (1992). Tight control of gene expression in mammalian cells by tetracycline-responsive promoters. Proc. Natl. Acad. Sci. USA 89, 5547-5551.
Gossen, M., Freundlieb, S., Bender, G., Muller, G., Hillen, W., & Bujard, H. (1995). Transcriptional activation by tetracyclines in mammalian cells. Science 268, 1766-1769.
Harding, T. C., Geddes, B. J., Murphy, D., Knight, D. & Uney, J. B. (1998) Switching transgene expression in the brain using an adenoviral tetracycline-regulatable system. Nat. Biotechnol. 16, 553-555.
Helbl, V., & Hillen, W. (1998). Stepwise selection of TetR variants recognizing tet operator 4C with high affinity and specificity. J. Mol. Biol. 276, 313-318.
Helbl, V., Tiebel, B., & Hillen, W. (1998). Stepwise selection of TetR variants recognizing tet operator 6C with high affinity and specificity. J. Mol. Biol. 276, 319-324.
Hillen, W., & Berens, C. (1994). Mechanisms underlying expression of tn10 encoded tetracycline resistance. Annu. Rev. Microbiol. 48, 345-369.
Hinrichs, W., Kisker, C., Düvel, M., Müller, A., Tovar, K., Hillen, W., & Saenger, W. (1994). Structure of the Tet repressor-tetracycline complex and regulation of antibiotic resistance. Science 264, 418-420.
Ito, H., Fukuda, Y., Murata, K., & Kimura, A. (1983). Transformation of intact yeast cells treated with alkali cations. J. Bact. 153, 163-168.
Kisker, C., Hinrichs, W., Tovar, K., Hillen, W., & Saenger, W. (1995). The complex formed between tet repressor and tetracycline-Mg²⁺ reveals mechanism of antibiotic resistance. J. Mol. Biol. 247, 260-280.
Kistner, A., Gossen, M., Zimmermann, F., Jerecic, J., Ullmer, C., Lübbert, H., & Bujard, H. (1996). Doxycycline-mediated quantitative and tissue-specific control of gene expression in transgenic mice. Proc. Natl. Acad. Sci. USA 93, 10933-10938.
Leung, D. W., Chen, E., & Goeddel, D. V. (1989). A method for random mutagenesis of a defined DNA segment using a modified polymerase chain reaction. Technique 1, 11-15.
Müller, G., Hecht, B., Helbl, V., Hinrichs, W., Saenger, W., & Hillen, W. (1995). Characterization of non-inducible Tet repressor mutants suggests conformational changes necessary for induction. Nature Struct. Biol. 2, 693-703.
Niedenthal, R. K., Riles, L., Johnston, M., & Hegemann, J. H. (1996). Green fluorescent protein as a marker for gene expression and subcellular localization in budding yeast. Yeast 12, 773-786.
Oldenburg, K. R., Vo, K. T., Michaelis, S., & Paddon, C. (1997). Recombination-mediated PCR-directed plasmid construction in vivo in yeast. Nucl. Acids Res. 25, 2.
Orth, P., Cordes, F., Schnappinger, D., Hillen, W., Saenger, W., & Hinrichs, W. (1998). Conformational changes of the Tet repressor induced by tetracycline trapping. J. Mol. Biol. 279, 439-447.
Pan, W., Ravot, E., Tolle, R., Frank, R., Mosbach, R., Türbachova, I. & Bujard, H. (1999) Vaccine candidate MSP-1 from Plasmodium falciparum: a redesigned 4917 bp polynucleotide enables synthesis and isolation of full length protein from E. coli and mammalian cells. Nucl. Acids Res. 27, 1094-1103.
Paulus, W., Baur, I., Oberer, D. M., Breakefield, X. O., & Reeves, S. A. (1997). Regulated expression of the diphtheria toxin A gene in human glioma cells using prokaryotic transcriptional control elements. J. Neurosurg. 87, 89-95.
Schnappinger, D., Schubert, P., Pfleiderer, K., & Hillen, W. (1998). Determinants of protein-protein recognition by four helix bundles: changing the dimerization specificity of Tet repressor. EMBO J. 17, 535-543.
Shockett, P., Difilippantonio, M., Hellman, N., & Schatz, D. G. (1995). A modified tetracycline-regulated system provides autoregulatory, inducible gene expression in cultured cells and transgenic mice. Proc. Natl. Acad. Sci. USA 92, 6522-6526.
Wach, A., Brachat, A., Alberti-Segui, C., Rebischung, C., & Philippsen, P. (1997). Heterologous HIS3 marker and GFP reporter modules for PCR-targeting in Saccharomyces cerevisiae. Yeast 13, 1065-1075.
Weinmann, P., Gossen, M., Hillen, W., Bujard, H. & Gatz, C. (1994) A chimeric transactivator allows tetracycline-responsive gene expression in whole plants. Plant J. 5, 559-569.
Zeidler, M., Gatz, C., Hartmann, E. & Hughes, J. (1996) Tetracycline-regulated reporter gene expression in the moss Physcomitrella patens. Plant Mol. Biol. 30, 199-205.
Zhou, Y., Zhang, X., & Ebright, R. H. (1991). Random mutagenesis of gene-sized DNA molecules by use of PCR with Taq DNA polymerase. Nucl. Acids Res. 19, 6052.
Gallego, C., Gari, E.,Colomina, N., Herrero E. and Aldea, M. (1997) The Cln3 cyclin is down-regulated by translational repression and degradation during the G1 arrest caused by nitrogen deprivation in budding yeast. EMBO Journal 16, 7196-7206.
Gan, E., Piedrafita, L., Aldea, M. and Herrero, E. (1997) A set of vectors with a tetracycline-regulatable promoter system for modulated gene expression in Saccharomyces cerevisiae. Yeast 13, 837-848.
Belli, G., Gari, E., Aldea, M. and Herrero, E. (1998a) Functional analysis of yeast essential genes using a promoter-substitution cassette and the tetracycline-regulatable dual expression system. Yeast 14, 1127-1138.
Belli, G., Gari, E., Piedrafita, L., Aldea, M. and Herrero, E. (1998b) An activator/repressor dual system allows tight tetracycline-regulated gene expression in budding yeast. Nucl. Acids Res. 26, 942-947.
Nagahashi, S., Lussier, M. and Bussey, H. (1998) Isolation of Candida glabrata homologs of the Saccharomyces cerevisiae KRE9 and KNH1 genes and their involvement in cell wall beta-1, 6-glucan synthesis. J. Bacteriol. 180, 5020-5029.
Nakayama, H., Izuta, M., Nagahashi, S., Sihta, E.Y., Sato, Y. Yamazaki, T., Arisawa, M. and Kitada, K. (1998) A controllable gene expression system for the pathogenic fungus Candida glabrata. Microbiology 144, 2407-2415.
Colomina, N., Gari, E., Gallego, C., Herrero, E. and Aldea, M. (1999) G1 cyclins block the Imel pathway to make mitosis and meiosis incompatible in budding yeast. EMBO J. 18, 320-329.
Wissmann, A., Wray, L. V. Jr., Somaggio, U., Baumeister, R., Geissendoerfer, M. and Hillen, W. (1991) Selection for Tn10 Tet repressor binding to tet operator in Escherichia coli: Isolation of temperature sensitive mutants and combinatorial mutagenesis in the DNA binding motif. Genetics 128, 225-232.
   Incorporation by Reference

All patents, published patent applications and other references disclosed herein are hereby expressly incorporated herein in their entireties by reference.

Equivalents

Those skilled in the art will recognize, or be able to ascertain using no more than routine experimentation, many equivalents to the specific embodiments of the invention described herein. Such equivalents are intended to be encompassed by the following claims.

1. A method for identifying a transcriptional regulatory protein that modulates transcription of a gene of interest, said method comprising:
providing a cell comprising a nucleic acid molecule comprising first, second and third polynucleotides, wherein:
said first polynucleotide encodes a fusion protein, said fusion protein comprising a first polypeptide in operative linkage to a second polypeptide, wherein said first polypeptide is derived from the DNA binding domain of a first protein that binds to a regulatory sequence of said gene of interest, and said second polypeptide is derived from the transcriptional regulatory domain of a second protein;
said second polynucleotide comprises an indicator gene, the expression of which is responsive to said fusion protein; and
said third polynucleotide comprises a selectable marker gene; and
detecting a signal generated by said indicator gene to thereby identify said fusion protein as a transcriptional regulatory protein that modulates transcription of said gene of interest.

2. The method of claim 1, wherein said cell is further treated with a modulator molecule, or analog thereof, wherein said binding of said first polypeptide to said regulatory sequence of said gene of interest is controlled by said modulator molecule, or an analog thereof.

3. The method of claim 2, wherein said first polypeptide is derived from the DNA binding domain of a Tet repressor protein.

4. The method of claim 1, wherein said second polypeptide activates transcription.

5. The method of claim 1, wherein said second polypeptide inhibits transcription.

6. The method of claim 3, wherein said second polypeptide is derived from Herpes simplex virion protein 16.

7. The method of claim 2, wherein binding of said first polypeptide to said regulatory sequence of said gene of interest is inhibited in the presence of said modulator molecule, or analog thereof.

8. The method of claim 2, wherein binding of said first polypeptide to said regulatory sequence of said gene of interest is dependent upon the presence of said modulator molecule, or analog thereof.

9. The method of claim 3, wherein binding of said first polypeptide to said regulatory sequence of said gene of interest is inhibited in the presence of said modulator molecule.

10. The method of claim 3, wherein binding of said first polypeptide to said regulatory sequence of said gene of interest is dependent upon the presence of an analog of said modulator molecule.

11. The method of claim 9, wherein said modulator molecule is tetracycline.

12. The method of claim 11, wherein said fusion protein comprises a tetracycline controlled transactivator (tTA) protein.

13. The method of claim 10, wherein said analog of said modulator molecule is selected from doxycycline, anhydrotetracycline, oxy-tetracycline, and chloro-tetracycline.

14. The method of claim 13, wherein said fusion protein comprises a reverse tetracycline controlled transactivator (rtTA) protein

15. The method of claim 1, wherein said first polynucleotide comprises a variant allele encoding said DNA binding domain of said first protein.

16. The method of claim 15, wherein said first protein is the Tet repressor protein.

17. The method of claim 16, wherein said fusion protein comprises a sequence variant of a tTA protein.

18. The method of claim 16, wherein said fusion protein comprises a sequence variant of a rtTA protein.

19. The method of claim 2, wherein the regulatory sequence of said gene of interest is derived from the Tet operator.

20. The method of claim 1, wherein the signal generated by the indicator gene is selected from a growth signal, an optical signal, and second messenger production.

21. The method of claim 20, wherein said indicator gene encodes green fluorescent protein.

22. The method of claim 1, wherein said selectable marker gene is selected from a gene that confers amino acid or nucleotide prototrophy, a gene that confers antibiotic resistance, and a gene that confers metabolic drug resistance.

23. The method of claim 22, wherein the selectable marker gene is URA3.

24. The method of claim 1, wherein the cell is selected from a prokaryotic cell, and a eukaryotic cell.

25. The method of claim 1, wherein the cell is a yeast cell.

26. The method of claim 25, wherein said cell is a yeast cell is of the species Saccharomyces cerevisiae.

27. The method of claim 1, wherein the cell is a mammalian cell.

28. A method for identifying a Tet repressor-based regulatory protein that modulates transcription of a gene of interest, said method comprising:
providing a cell comprising a nucleic acid molecule comprising first, second and third polynucleotides, wherein:
said first polynucleotide encodes a Tet repressor-based regulatory protein, said Tet repressor-based regulatory protein comprising a first polypeptide in operative linkage to a second polypeptide, wherein said first polypeptide is derived from a Tet repressor protein that binds to a regulatory sequence of said gene of interest, wherein said binding to said regulatory sequence is controlled by a modulator molecule, or an analog thereof, and said second polypeptide is derived from the transcriptional regulatory domain of a second protein;
said second polynucleotide comprises an indicator gene, the expression of which is responsive to said Tet repressor-based regulatory protein; and
said third polynucleotide comprises a selectable marker gene;
treating said cell with said modulator molecule, or an analog thereof; and
detecting a signal generated by said indicator gene to thereby identify said Tet repressor-based regulatory protein as a modulator of transcription of said gene of interest.

29. The method of claim 28, wherein said modulator molecule is tetracycline.

30. The method of claim 29, wherein said Tet repressor-based regulatory protein comprises a tetracycline controlled transactivator (tTA) protein.

31. The method of claim 28, wherein said analog of said modulator molecule is selected from doxycycline, anhydrotetracycline, oxy-tetracycline, and chloro-tetracycline.

32. The method of claim 31, wherein said Tet repressor-based regulatory protein comprises a reverse tetracycline controlled transactivator (rtTA) protein.

33. The method of claim 28, wherein said second polypeptide activates transcription.

34. The method of claim 28, wherein said second polypeptide inhibits transcription.

35. The method of claim 33, wherein said second polypeptide is derived from Herpes simplex virion protein 16.

36. The method of claim 28, wherein said first polynucleotide comprises a variant allele of said Tet repressor-based regulatory protein.

37. The method of claim 36, wherein said Tet repressor-based regulatory protein comprises a sequence variant of a tTA protein.

38. The method of claim 36, wherein said Tet repressor-based regulatory protein comprises a sequence variant of a rtTA protein.

39. The method of claim 28, wherein the regulatory sequence of said gene of interest is derived from the Tet operator.

40. The method of claim 28, wherein the signal generated by the indicator gene is selected from a growth signal, an optical signal, and second messenger production.

41. The method of claim 40, wherein said indicator gene encodes green fluorescent protein.

42. The method of claim 28, wherein said selectable marker gene is selected from a gene that confers amino acid or nucleotide prototrophy, a gene that confers antibiotic resistance, and a gene that confers metabolic drug resistance.

43. The method of claim 42, wherein the selectable marker gene is URA3.

44. The method of claim 28, wherein the cell is selected from a prokaryotic cell, or a eukaryotic cell.

45. The method of claim 28, wherein said cell is a yeast cell.

46. The method of claim 45, wherein said cell is a yeast cell is of the species Saccharomyces cerevisiae.

47. A method for identifying a Tet repressor-based regulatory protein that modulates transcription, said method comprising:
providing a cell comprising a nucleic acid molecule that comprises first, second and third polynucleotides, wherein:
said first polynucleotide encodes a Tet repressor-based regulatory protein, said Tet repressor-based regulatory protein comprising a first polypeptide in operative linkage to a second polypeptide, wherein said first polypeptide is derived from a Tet repressor protein that binds to regulatory sequences derived from the Tet operator, wherein said binding is controlled by tetracycline, or an analog thereof, and said second polypeptide is derived from the transcription activation domain of a second protein; and
said second polynucleotide comprises an indicator gene, the expression of which is regulated by sequences derived from the Tet operator; and
said third polynucleotide comprises a selectable marker gene;
treating said cell with tetracycline, or an analog thereof; and
detecting a signal generated by said indicator gene to thereby identify said Tet repressor-based regulatory protein as a modulator of transcription.

48. The method of claim 47, wherein said Tet repressor-based regulatory protein comprises a tetracycline controlled transactivator (tTA) protein.

49. The method of claim 47, wherein said tetracycline analog is selected from doxycycline, anhydrotetracycline, oxy-tetracycline, and chloro-tetracycline.

50. The method of claim 49, wherein said Tet repressor-based regulatory protein comprises a reverse tetracycline controlled transactivator (rtTA) protein.

51. The method of claim 48, wherein said Tet repressor-based regulatory protein comprises a sequence variant of a tTA protein.

52. The method of claim 50, wherein said Tet repressor-based regulatory protein comprises a sequence variant of a rtTA protein.

53. The method of claim 47, wherein said transcription activation domain is derived from Herpes simplex virion protein 16.

54. The method of claim 53, wherein said transcription activation domain comprises at least one copy of a minimal activation domain.

55. The method of claim 47, wherein the signal generated by the indicator gene is selected from a growth signal, an optical signal, and second messenger production.

56. The method of claim 55, wherein said indicator gene encodes green fluorescent protein.

57. The method of claim 47, wherein said selectable marker gene is selected from a gene that confers amino acid or nucleotide prototrophy, a gene that confers antibiotic resistance, and a gene that confers metabolic drug resistance.

58. The method of claim 57, wherein the selectable marker gene is URA3.

59. The method of claim 47, wherein the cell is selected from a prokaryotic cell, or a eukaryotic cell.

60. The method of claim 47, wherein the cell is a yeast cell.

61. The method of claim 60, wherein said cell is a yeast cell is of the species Saccharomyces cerevisiae.

62. A method for identifying a compound that is capable of modulating a transcriptional regulatory protein that modulates transcription of a gene of interest, said method comprising:
providing a cell comprising a nucleic acid molecule comprising first, second, and third polynucleotides, wherein:
said first polynucleotide encodes a fusion protein, said fusion protein comprising a first polypeptide in operative linkage to a second polypeptide, wherein said first polypeptide is derived from the DNA binding domain of a first protein that binds to a regulatory sequence of said gene of interest, wherein said binding to said regulatory sequence is controlled by a modulator compound, and said second polypeptide is derived from a transcription activation domain of a second protein; and
said second polynucleotide comprises an indicator gene, the expression of which is responsive to said fusion protein; and
said third polynucleotide comprises a selectable marker gene;
treating said cell with said compound; and
detecting a signal generated by said indicator gene to thereby identify said compound as a modulator of said transcriptional regulatory protein.

63. The method of claim 62, wherein said gene of interest is tet A.

64. The method of claim 62, wherein said first polypeptide is derived from the Tet-repressor protein and binds to the DNA binding domain of the Tet operator.

65. The method of claim 62, wherein said fusion protein comprises a tetracycline controlled transactivator (tTA) protein.

66. The method of claim 62, wherein said fusion protein comprises a reverse tetracycline controlled transactivator (rtTA) protein.

67. The method of claim 65, wherein said Tet repressor-based regulatory protein comprises a sequence variant of a tTA protein.

68. The method of claim 66, wherein said Tet repressor-based regulatory protein comprises a sequence variant of a rtTA protein.

69. The method of claim 62, wherein said cell is treated with a compound selected from a library of test compounds.

70. The method of claim 62, wherein said transcription activation domain is derived from Herpes simplex virion protein 16.

71. The method of claim 62, wherein the signal generated by the indicator gene is selected from a growth signal, an optical signal, and second messenger production.

72. The method of claim 71, wherein said indicator gene encodes green fluorescent protein.

73. The method of claim 62, wherein said selectable marker gene is selected from a gene that confers amino acid or nucleotide prototrophy, a gene that confers antibiotic resistance, and a gene that confers metabolic drug resistance.

74. The method of claim 73, wherein the selectable marker gene is URA3.

75. The method of claim 62, wherein the cell is selected from a prokaryotic cell, or a eukaryotic cell.

76. The method of claim 62, wherein the cell is a yeast cell.

77. The method of claim 76, wherein said cell is a yeast cell is of the species Saccharomyces cerevisiae.

78. A recombinant vector comprising:
a nucleic acid molecule comprising first, second and third polynucleotides, wherein:
said first polynucleotide encodes a fusion protein, said fusion protein comprising a first polypeptide in operative linkage to a second polypeptide, wherein said first polypeptide is derived from the DNA binding domain of a first protein that binds to a regulatory sequence of said gene of interest, and said second polypeptide is derived from the transcriptional regulatory domain of a second protein;
said second polynucleotide comprises an indicator gene, the expression of which is responsive to said fusion protein; and
said third polynucleotide comprises a selectable marker gene.

79. A recombinant vector comprising:
a nucleic acid molecule comprising first, second and third polynucleotides, wherein:
said first polynucleotide encodes a Tet repressor-based regulatory protein, said Tet repressor-based regulatory protein comprising a first polypeptide in operative linkage to a second polypeptide, wherein said first polypeptide is derived from a Tet repressor protein that binds to a regulatory sequence of said gene of interest, wherein said binding to said regulatory sequence is controlled by a modulator molecule, or an analog thereof, and said second polypeptide is derived from the transcriptional regulatory domain of a second protein; and
said second polynucleotide comprises an indicator gene, the expression of which is responsive to said Tet repressor-based regulatory protein; and
said third polynucleotide comprises a selectable marker gene.

80. A recombinant vector comprising:
a nucleic acid molecule that comprises first, second and third polynucleotides, wherein:
said first polynucleotide encodes a Tet repressor-based regulatory protein, said Tet repressor-based regulatory protein comprising a first polypeptide in operative linkage to a second polypeptide, wherein said first polypeptide is derived from a Tet repressor protein that binds to regulatory sequences derived from the Tet operator, wherein said binding is controlled by tetracycline, or an analog thereof, and said second polypeptide is derived from the transcription activation domain of a second protein; and
said second polynucleotide comprises an indicator gene, the expression of which is regulated by sequences derived from the Tet operator; and
said third polynucleotide comprises a selectable marker gene.

81. A host cell transformed with a recombinant vector of claim 78, 79, or 80.

82. A host cell of claim 81, wherein the cell is selected from a prokaryotic cell, a eukaryotic cell, a yeast cell, and a mammalian cell.

83. A method for identifying a polynucleotide gene regulatory sequence that binds to a Tet repressor-based regulatory protein of interest, said method comprising:
providing a cell comprising a nucleic acid molecule that comprises first, second and third polynucleotides, wherein:
said first polynucleotide encodes a Tet repressor-based regulatory protein, said Tet repressor-based regulatory protein comprising a first polypeptide in operative linkage to a second polypeptide, wherein said first polypeptide is derived from a Tet repressor protein that binds to gene regulatory sequences derived from the Tet operator, wherein said binding is controlled by tetracycline, or an analog thereof, and said second polypeptide is derived from the transcription activation domain of a second protein;
said second polynucleotide comprises an indicator gene, the expression of which is modulated by gene regulatory sequences derived from the Tet operator; and
said third polynucleotide comprises a selectable marker gene;
treating said cell with tetracycline, or an analog thereof; and
detecting a signal generated by said indicator gene to thereby identify said gene regulatory sequences as binding said Tet repressor-based regulatory protein and modulating gene transcription.

84. The method of claim 83, wherein said gene regulatory sequences comprise Tet operator sequence variants having at least one nucleotide substitution.

85. The method of claim 83, wherein said Tet repressor-based regulatory protein comprises a tetracycline controlled transactivator (tTA) protein.

86. The method of claim 83, wherein said tetracycline analog is selected from doxycycline, anhydrotetracycline, -oxy-tetracycline, and chloro-tetracycline.

87. The method of claim 86, wherein said Tet repressor-based regulatory protein comprises a reverse tetracycline controlled transactivator (rtTA) protein.

88. The method of claim 85, wherein said Tet repressor-based regulatory protein comprises a sequence variant of a tTA protein.

89. The method of claim 87, wherein said Tet repressor-based regulatory protein comprises a sequence variant of a rtTA protein.

90. The method of claim 83, wherein said transcription activation domain is derived from Herpes simplex virion protein 16.

91. The method of claim 83, wherein the signal generated by the indicator gene is selected from a growth signal, an optical signal, and second messenger production.

92. The method of claim 91, wherein said indicator gene encodes green fluorescent protein.

93. The method of claim 83, wherein said selectable marker gene is selected from a gene that confers amino acid or nucleotide prototrophy, a gene that confers antibiotic resistance, and a gene that confers metabolic drug resistance.

94. The method of claim 93, wherein the selectable marker gene is URA3.

95. The method of claim 83, wherein the cell is selected from a prokaryotic cell, or a eukaryotic cell.

96. The method of claim 83, wherein the cell is a yeast cell.

97. The method of claim 96, wherein said yeast cell is of the species Saccharomyces cerevisiae.

98. A method for identifying a polynucleotide gene regulatory sequence that binds to a transcriptional regulatory protein of interest, said method comprising:
providing a cell comprising a nucleic acid molecule that comprises first, second and third polynucleotides, wherein:
said first polynucleotide encodes a fusion protein, said fusion protein comprising a first polypeptide in operative linkage to a second polypeptide, wherein said first polypeptide is derived from the DNA binding domain of a first protein that binds to gene regulatory sequences of a gene of interest, and said second polypeptide is derived from the transcriptional regulatory domain of a second protein;
said second polynucleotide comprises an indicator gene, the expression of which is modulated by said gene regulatory sequences of said gene of interest; and
said third polynucleotide comprises a selectable marker gene; and
detecting a signal generated by said indicator gene to thereby identify said gene regulatory sequences as binding said fusion protein and modulating gene transcription.

99. The method of claim 98, wherein said gene regulatory sequences comprise variants of the gene regulatory sequences of said gene of interest having at least one nucleotide substitution.

100. The method of claim 98, wherein said cell is further treated with a modulator molecule, or analog thereof, wherein said binding of said first polypeptide to said regulatory sequence of said gene of interest is controlled by said modulator molecule, or an analog thereof.

101. The method of claim 100, wherein said first polypeptide is derived from the DNA binding domain of a Tet repressor protein.

102. The method of claim 98, wherein said second polypeptide activates transcription.

103. The method of claim 98, wherein said second polypeptide inhibits transcription.

104. The method of claim 101, wherein said second polypeptide is derived from Herpes simplex virion protein 16.

105. The method of claim 100, wherein binding of said first polypeptide to said regulatory sequence of said gene of interest is inhibited in the presence of said modulator molecule, or analog thereof.

106. The method of claim 100, wherein binding of said first polypeptide to said regulatory sequence of said gene of interest is dependent upon the presence of said modulator molecule, or analog thereof.

107. The method of claim 101, wherein binding of said first polypeptide to said regulatory sequence of said gene of interest is inhibited in the presence of said modulator molecule.

108. The method of claim 101, wherein binding of said first polypeptide to said regulatory sequence of said gene of interest is dependent upon the presence of an analog of said modulator molecule.

109. The method of claim 107, wherein said modulator molecule is tetracycline.

110. The method of claim 109, wherein said fusion protein comprises a tetracycline controlled transactivator (tTA) protein.

111. The method of claim 108, wherein said analog of said modulator molecule is selected from doxycycline, anhydrotetracycline, oxy-tetracycline, and chloro-tetracycline.

112. The method of claim 111, wherein said fusion protein comprises a reverse tetracycline controlled transactivator (rtTA) protein

113. The method of claim 98, wherein said first polynucleotide comprises a variant allele encoding said DNA binding domain of said first protein.

114. The method of claim 113, wherein said first protein is the Tet repressor protein.

115. The method of claim 114, wherein said fusion protein comprises a sequence variant of a tTA protein.

116. The method of claim 114, wherein said fusion protein comprises a sequence variant of a rtTA protein.

117. The method of claim 100, wherein the regulatory sequence of said gene of interest is derived from the Tet operator.

118. The method of claim 117, wherein said gene regulatory sequences comprise Tet operator sequence variants having at least one nucleotide substitution.

119. The method of claim 98, wherein the signal generated by the indicator gene is selected from a growth signal, an optical signal, and second messenger production.

120. The method of claim 119, wherein said indicator gene encodes green fluorescent protein.

121. The method of claim 98, wherein said selectable marker gene is selected from a gene that confers amino acid or nucleotide prototrophy, a gene that confers antibiotic resistance, and a gene that confers metabolic drug resistance.

122. The method of claim 121, wherein the selectable marker gene is URA3.

123. The method of claim 98, wherein the cell is selected from a prokaryotic cell, and a eukaryotic cell.

124. The method of claim 98, wherein the cell is a yeast cell.

125. The method of claim 124, wherein said cell is a yeast cell is of the species Saccharomyces cerevisiae.

126. The method of claim 98, wherein the cell is a mammalian cell.
